(19) United States
(12) Patent Application Publication
(10) Pub. No.: US 2024/0203724 A1
(43) Pub. Date: Jun. 20, 2024

(54) SYSTEMS AND METHODS FOR SINGLE-ION MASS SPECTROMETRY WITH TEMPORAL INFORMATION

(71) Applicant: Brown University, Providence, RI (US)

(72) Inventors: Derek M. Stein, Providence, RI (US); Nicholas Drachman, Providence, RI (US); Benjamin Wiener, Somerville, MA (US)

(73) Assignee: Brown University, Providence, RI (US)

(21) Appl. No.: 18/287,052

(22) PCT Filed: Apr. 22, 2022
(86) PCT No.: PCT/US2022/025902

§ 371 Date: Oct. 16, 2023

Related U.S. Application Data
(60) Provisional application No. 63/179,046, filed on Apr. 23, 2021.

(51) Int. Cl. H01J 49/42; C12Q 1/6872; G01N 33/68; H01J 49/04; H01J 49/16; H01J 49/22; H01J 49/24

(52) U.S. Cl. CPC H01J 49/4215; C12Q 1/6872; G01N 33/6848; H01J 49/0404; H01J 49/164; H01J 49/22; H01J 49/24

(57) ABSTRACT

The present disclosure generally relates to mass spectrometers, including but not limited to mass spectrometers able to emit ions at determinable times. In some aspects, the time between when ions leave an ion source and the time the ions reach a detector may be determined at a relatively high time resolutions, which may be useful for certain applications such as sequencing of biopolymers. In addition, in some cases, a relatively high number of ions leaving an ion source may be determined at a detector, e.g., at least 50% or more of the ions that are produced. Other aspects are generally directed to systems and methods for using such mass spectrometers, techniques involving such mass spectrometers, or the like.

### RELATED APPLICATIONS

This application claims the benefit of U.S. Provisional Patent Application Ser. No. 63/179,046, filed Apr. 23, 2021, entitled “Systems and Methods for Single-Ion Mass Spectrometry with Temporal Information,” by Stein, et al., incorporated herein by reference in its entirety.

### FIELD

The present disclosure generally relates to mass spectrometers, including but not limited to mass spectrometers able to emit ions at determinable times.

### BACKGROUND

Mass spectrometry could be well suited for sequencing proteins because it is an analytical technique with the ability to identify all 20 amino acids. Typically, mass spectrometers measure ions using a single ion detector and a mass filter that sweeps a narrow mass transmission window in time. For example, a quadrupole mass filter allows only the ions within a narrow m/z range to travel through it, with the particular m/s range controlled by the time-varying voltages applied to the four poles of the quadrupole. As the allowed m/z range is swept, the ion transmission rate is measured by the detector, and mass spectra are determined after at least one sweep. Ions with an m/z outside the transmission window do not pass through the filter. Therefore, a particular ion leaving the source will only be detected and identified if it happens to pass through the filter at the moment when the window is centered on that ion's m/z, and that cannot be guaranteed without knowing the order of ion m/z values in advance. Accordingly, such systems cannot be used for sequencing, as it is not known when the ion left the ion source, and in what order. Thus, mass spectrometers have not been able to be used to sequence single proteins.

### SUMMARY

The present disclosure generally relates to mass spectrometers, including but not limited to mass spectrometers able to emit ions at determinable times. The subject matter of the present disclosure involves, in some cases, interrelated products, alternative solutions to a particular problem, and/or a plurality of different uses of one or more systems and/or articles.

One aspect is generally directed to a mass spectrometer. In one set of embodiments, the mass spectrometer comprises an ion source comprising a capillary and an electrode proximate the capillary, a magnetic mass filter downstream of the ion source, and an array of detectors downstream of the magnetic mass filter. In some cases, the capillary comprises an opening having a cross-sectional dimension of less than 125 nm.

In another set of embodiments, the mass spectrometer, comprises an ion source constructed and arranged to produce single ions or ion clusters, a magnetic mass filter positioned to receive the single ions or ion clusters from the ion source, a pump able to create a pressure less than 100 mPa in an environment positioned between the ion source and the magnetic mass filter, and an array of detectors positioned to receive the single ions or ion clusters from the magnetic mass filter.

The mass spectrometer, in still another set of embodiments, comprises an ion source, a magnetic mass filter downstream of the ion source, and an array of detectors downstream of the magnetic mass filter.

Another aspect is generally directed towards a method of sequencing a biopolymer. According to one set of embodiments, the method comprises ionizing a biopolymer contained within a fluid into ions or ion clusters, passing the ions or ion clusters through a magnetic mass filter, directing the ions or ion clusters to an array of detectors, and determining a sequence of the biopolymer by determining the ions or ion clusters with the array of detectors.

The method, in another set of embodiments, comprises passing a fluid comprising a biopolymer into a capillary defining an opening, applying an electric field to ionize the biopolymer proximate the opening to produce ions or ion clusters, passing the ions or ion clusters directly into an environment having a pressure of no more than 100 mPa, passing the ions or ion clusters through a magnetic mass filter, directing the ions or ion clusters to an array of detectors, and determining a sequence of the biopolymer by determining the ions or ion clusters with the array of detectors.

Yet another aspect is generally directed towards a method of determining a concentration. In one set of embodiments, the method comprises ionizing molecules from a fluid as ions or ion clusters, passing the ions or ion clusters through a magnetic mass filter, directing the ions or ion clusters to an array of detectors, and determining a concentration of the molecules in the fluid by determining the ions or ion clusters with the array of detectors.

Still another aspect is directed to a method comprising ionizing molecules from a fluid as ions or ion clusters, passing at least 50% of the ions or ion clusters through a magnetic mass filter, and directing the ions or ion clusters to a detector.

Another aspect is directed to a method comprising ionizing molecules from a fluid as ions or ion clusters using an ion source, passing the ions or ion clusters through a mass filter, directing the ions or ion clusters to a detector, and determining a duration between the time the ions or ion clusters leave the ion source and the time the ions or ion clusters arrives at the detector.

Yet another aspect is directed to a method comprising ionizing molecules using an ion source to produce a sequence of ions or ion clusters, passing the sequence of ions or ion clusters through a mass filter, and directing the sequence of ions or ion clusters to an array of detectors. In some cases, at least 50% of the ions or ion clusters arriving at the array of detectors arrive in sequence. In addition, in some cases, at least 90% of the ions or ion clusters arriving at the array of detectors arrive in sequence.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a nanopore mass spectrometer, in accordance with one embodiment;

FIGS. 2A-2B show mass spectra of positive amino acid ions delivered directly into high vacuum from a nanopore ion source, in another embodiment;

FIG. 3 is a schematic drawing of a mass spectrometer in accordance with still another embodiment;

FIGS. 4A-4D illustrate operation of nanopore mass spectrometers, in accordance with certain embodiments;

FIGS. 5A-5C illustrate the mass spectra of certain biomolecules, in accordance with some embodiments;

FIGS. 6A-6E illustrate the production or emission of ions from an ion source, in yet other embodiments; and

FIG. 7 illustrates a magnetic mass filter, in accordance with still another embodiment.

### DETAILED DESCRIPTION

The present disclosure generally relates to mass spectrometers, including but not limited to mass spectrometers able to emit ions at determinable times. In some aspects, the time between when ions leave an ion source and the time the ions reach a detector may be determined at a relatively high time resolutions, which may be useful for certain applications such as sequencing of biopolymers. In addition, in some cases, a relatively high number of ions leaving an ion source may be determined at a detector, e.g., at least 50% or more of the ions that are produced. Other aspects are generally directed to systems and methods for using such mass spectrometers, techniques involving such mass spectrometers, or the like.

Conventional mass spectrometers typically measure ions using a single ion detector and a mass filter that sweeps a narrow mass transmission window in time. For example, as the allowed mass-to-charge ratio range is swept, only ions with a mass-to-charge ratio inside the allowed range can be measured. As such, conventional mass spectrometers are capable of only determining the mass-to-charge ratio of a fraction of the emitted ions. Furthermore, the capabilities of conventional mass spectrometers may be rather limited. For example, conventional mass spectrometers cannot be used to provide valuable information about ion orderings, ion associations, and/or ions sequences. Accordingly, certain aspects of the disclosure are directed to a mass spectrometer that can be used to provide such information.

In one example, the mass spectrometer comprises a particular combination of components and/or configuration that imparts the spectrometer enhanced detection and measurement capabilities. For instance, the mass spectrometer may comprise an ion source capable of ionizing molecules into single ions, a magnetic mass filter capable of sorting ions based on their mass-to-charge ratios, and an array of detectors capable of determining the single ions. The combination of these features may advantageously allow for the determination of the sequence, structure, and/or identity of a species of interest, e.g., a biopolymer. For instance, the mass spectrometer may be used to sequence a protein or a nucleic acid. Advantageously, compared to convention mass spectrometers, the mass spectrometer described herein may have a high temporal resolution (e.g., less than 1 microsecond) and a high overall ion transmission efficiency (e.g., greater than or equal to about 0.8).

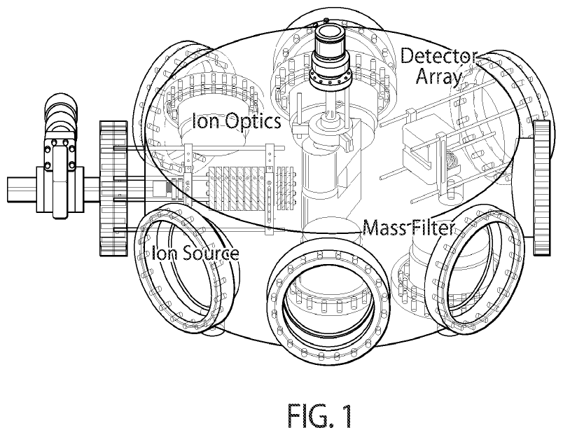

One non-limiting example of a mass spectrometer is shown in FIG. 1. In this example, ions (or ion clusters) are produced from an ion source that, in some cases, is able to produce ions at determinable times. The ion source may comprise a capillary tip that may allow for direct ion evaporation of samples with an applied electric field. In some cases, the tip may have an opening with a cross-section less than 100 nm. Examples of such systems can be seen in a PCT application filed on even date herewith, entitled “Nanotip Ion Sources and Methods,” as well as U.S. Pat. Apl. Ser. No. 63/015,407, filed Apr. 24, 2020, entitled “Nanotip Ion Sources and Methods,” each of which is incorporated herein by reference in its entirety. Fluid enters the ion source via a fluid inlet and molecules within the fluid are converted into ions or ion clusters in the ion source. In addition, ions (or ion clusters) leaving the ion source may optionally pass through an ion lens or other suitable ion optics, e.g., to focus the ions, and enter a chamber with a relatively low pressure environment (a “vacuum” chamber), for example, a pressure of less than 100 mPa (absolute). In some embodiments, the single ions or ion clusters from the ion source may be directly emitted into the vacuum or low pressure environment. In some embodiments, the mass spectrometer comprises a pump that is used create such a vacuum or low pressure environment.

A mass filter may then be used to sort the ions, for example, based on the masses or mass-to-charge ratios. In some embodiments, the mass filter (e.g., a magnetic mass filter) may be downstream of the ion source. The mass filter may be positioned to receive the single ions or ion clusters from the ion source. One non-limiting example is a magnetic sector mass filter. In some cases, ions or ion clusters with different mass-to-charge ratios may be deflected at different degrees due to the magnetic field created by the magnetic mass filter. Accordingly, upon exiting the magnetic mass filter, ions with different masses or mass-to-charge ratios may have different trajectories.

The ions or ion filters, upon exiting the magnetic mass filter, may be directed towards a detector array, e.g., positioned downstream of the mass filter (e.g., the magnetic mass filter). The array of detectors may be arranged and constructed to detect the single ions or ion clusters passed from the mass filter. Since the ions may not necessarily have the same trajectories, an array of detectors can be used, positioned to receive ions having different trajectories. Each detector may detect ions having certain masses or mass-to-charge ratios, e.g., with respect to time; the position of the detector is related to the incident masses or mass-to-charge ratios of the ions reaching the detector. Thus, since the time that the ions or ion clusters left the ion source is known, and there are no substantial collisions (e.g., with air molecules) between the ion source and the detector array, the time of travel between these can be determined, e.g., with relatively high time resolutions, for example, microseconds or less. In contrast, mass spectrometry systems that include air and/or time-varying voltages cannot achieve such time resolutions or perform sequencing.

Accordingly, in some embodiments, a mass spectrometer as described herein may have a relatively high temporal resolution. In some embodiments, the mass spectrometer has a temporal resolution of less than or equal to 1 microsecond (less than or equal to 500 nanoseconds, less than or equal to 250 nanoseconds, less than or equal to 100 nanoseconds, less than or equal to 50 nanoseconds, less than or equal to 10 nanoseconds, etc.).

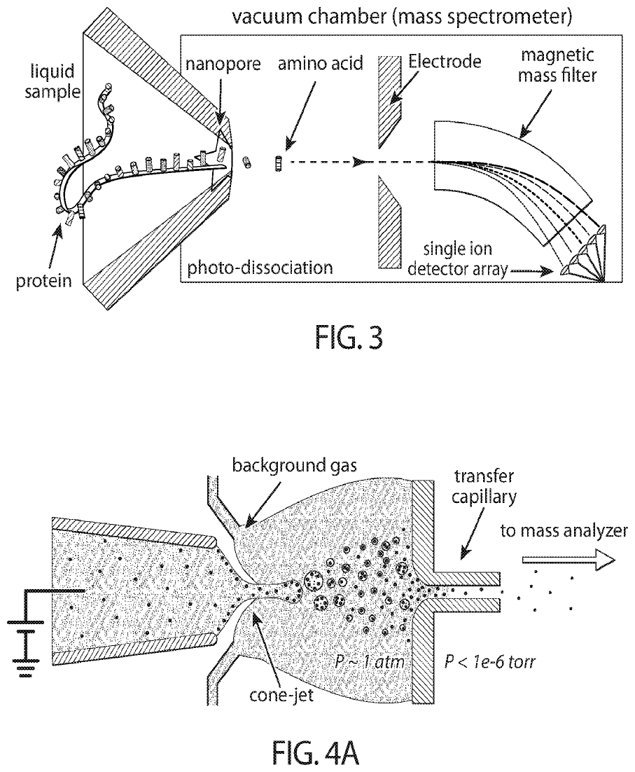

In some cases, information regarding the time of travel of ions can be used for certain types of sequencing, e.g., where monomers forming a polymer, such as amino acids or nucleotides in a protein or a nucleic acid, can be sequentially ionized and passed to the detectors, and such information used to reconstruct or “sequence” the original polymer. FIG. 3 illustrates another non-limiting embodiment of a mass spectrometer, e.g., for sequencing a polymer, such as a protein in this example. As shown, a mass spectrometer comprises an ion source (e.g., having a nanopore) capable of emitting single ions and ion clusters into a vacuum chamber, an electrode proximate the ion source, a magnetic mass filter downstream the ion source, and an array of detectors (e.g., single-ion detectors) downstream the magnetic mass filter.

In addition, in some embodiments, relatively high numbers of ions or ion clusters produced by the ion source may pass through the magnetic mass filter to the detector, e.g., due to the relatively low pressures present within the mass spectrometer. In some cases, at least 50% or more of the ions that are produced may reach one of the detectors. This can also be useful, for example, to determine concentrations of certain ionizable molecules within the fluid.

Thus, certain aspects of the disclosure are directed to systems and methods related to a mass spectrometer. In some embodiments, the mass spectrometer comprises an ion source constructed and arranged to produce single ions or ion clusters. An ion cluster may refer comprise a single ion and a number of solvent molecules. As an example, an ion cluster may include an ion with only 1 or 2 solvent molecules (for example, water).

The ion source may be any of a variety of ion sources, e.g., that are capable of ionizing a species of interest (e.g., a biopolymer) into single ions or ion clusters. In one set of embodiments, for example, an ion source comprising a capillary and an electrode proximate the capillary is described herein. The capillary comprises an opening having a cross-sectional dimension of less than 125 nm (e.g., less than 100 nm, less than 60 nm, etc.). In some embodiments, the electrode may be used to apply an electric field to a fluid within the capillary, such that molecules from a species of interest within the fluid may be ionized as single ions or ion clusters. Specific configurations and components of the ion source are disclosed in more detail below.

For example, in some embodiments, the ion source may comprise a capillary and an electrode, which may be annular in some cases, between which a voltage is applied to produce ions. In some cases, the capillary may have an inner tip diameter of less than 125 nm or less than 100 nm. This may allow ions to evaporate directly off of the meniscus of a fluid in the capillary, bypassing the wasteful droplet evaporation process. In this regime, ion evaporation may account for the majority of the ionic current, and this emission mode can be achieved in some cases with relatively low salinity solutions. In some embodiments, tips with inner diameters less than 125 nm or less than 100 nm may be able to produce a high fraction of bare ions or ionic clusters, for example, comprising small numbers of solvent molecules, e.g., only 1 or 2 solvent molecules. The small area of the liquid vacuum interface may in some cases prevent significant evaporative heat loss, which allows the use of volatile solvents like water in certain cases. Methods such as these could be used in some embodiments to analyze molecules or ions, e.g., biomolecules such as amino acids, nucleic acids, peptides or proteins, etc. In some cases, ion sources such as those described herein may improve the sensitivity of mass spectrometry experiments, allow single-molecule protein sequencing or single cell proteomic studies. Other applications such as those described below are also possible.

For example, some embodiments are generally directed to an ion source comprising a capillary and an electrode. The electrode may be used to generate ionized molecules directly from a fluid within the capillary, e.g., into a reduced pressure environment or vacuum, e.g., at a pressure of 100 mPa, or other pressures described herein. In certain embodiments, the opening of the capillary is sized such that, when an electric field is applied, a fluid within the capillary forms a charged meniscus and species within the fluid exit the charged meniscus, e.g., via predominately ion evaporation. The use of capillaries with a submicron opening (e.g., less than 100 nm) may favor the ionization of a fluid via ion evaporation, where the species exiting the capillary directly ionizes into single charged ions or charged ion clusters, in contrast to electrospray ionization, where the species exiting the capillary exit via a liquid jet that breaks up into charged droplets that further break down into charged ions in the presence of a background gas, although it should be understood that some electrospray ionization may still occur in some cases. Ion evaporation may be preferred in certain applications, e.g., that require the efficient use or generation of single ions from a fluid. For example, certain embodiments are related to ion sources in mass spectroscopy, where single charged ions can be directly generated and subsequently detected.

According to one set of embodiments, the ion source comprises a capillary defining an opening having a cross-sectional dimension (e.g., inner diameter of the capillary) of less than 100 nm. The opening may also be sized in some cases such that when an electric field is applied, ion evaporation dominates over liquid jet formation. For instance, in certain embodiments, at least 50% of the exiting species may exit via ion evaporation or in the form of ions or ion clusters. For instance, a nanoscale capillary can allow ions to evaporate directly off of a fluid meniscus. In some embodiments, a fluid can be passed into a capillary having such an opening, and directly delivered into a reduced pressure or vacuum environment (e.g., having a pressure of no more than 100 mPa) in the form of ions and ion clusters. The ions and ion clusters can be analyzed by a mass filter and an ion detector in a mass spectrometer, or applied to other applications such as those described herein.

It should be understood that other types of ion sources may also be employed. For example, the ion source may comprise a pulsed laser that is capable of ionizing molecules from a species of interest as single ions or ion clusters.

Certain embodiments comprise ionizing molecules contained within a fluid as ions or ion clusters. In some embodiments, the molecules may be ionized into single ions or ion clusters (i.e., single ion clustered with solvent molecules). In some embodiments, the molecules may be ionized as single ions with little, if any, of ion clusters present.

In one set of embodiments, the molecules contained within the fluid may be molecules from a polymer or a biopolymer (e.g., protein, polypeptides, nucleic acids, etc.). In another set of embodiments, the molecules may be small molecules (e.g., monomers, bio-monomers, salt ions, etc.) contained within the fluid that is capable of being ionized from the fluid.

The molecules may be ionized into single ions or ion clusters by any appropriate ion sources. In one set of embodiments, the molecules may be ionized from the fluid using an ion source described herein. For instance, the ion source may comprise a capillary comprising a relatively small opening. In some embodiments, a fluid comprising a species of interest (e.g., biopolymer) may be passed into the opening of the capillary. By applying an electric field to the fluid proximate the opening, molecules within the species of interest (e.g., biopolymer) may be ionized to produce single ions or ion clusters. Specific embodiments related to such an ion source are described below.

While the above embodiment described ion source comprising a capillary, it should be understood that any type of ion source may be employed in the mass spectrometer, as long as the ion source is capable of producing single ions or ion clusters. For instance, in one set of embodiments, the molecules may be ionized into single ions or ions clusters by a pulsed laser.

In some embodiments, a polymer such as a biopolymer contained within a fluid may be ionized into ions or ion clusters. Examples of biopolymers include, but are not limited to proteins, peptides, nucleic acids such as DNA or RNA, carbohydrates, polysaccharides, or the like. These may be ionized into monomeric components such as amino acids, nucleotides, sugar units or monosaccharides, etc. In some embodiments, upon ionization, single ions or ion clusters may be released from the biopolymer sequentially. For instance, in some cases, a single ion may be released from the biopolymer at a time. Advantageously, the ability to ionize a biopolymer into single ions or ion clusters and release them sequentially may reveal spatial and/or temporal information about the ordering or sequence of the ions in the molecule. For instance, a biopolymer may be ionized into a sequence of ions or ion clusters (e.g., ionized monomers) that corresponds to a sequence of base components (e.g., monomers) associated the biopolymer prior to ionization.

FIG. 3 illustrates a non-limiting example of such an embodiment. As shown, an ion source (e.g., nanopore) may be used to ionize a biopolymer (e.g., protein) into single ions (e.g., an amino acid) or ion clusters (e.g., amino acid with solvent molecules). The single ions may be released from the biopolymer in a sequential order as a sequence of ions or ion clusters. As shown, the sequence of ions released may correspond to a sequence of ions in the biopolymer prior to ionization.

In addition, the ions or ion clusters may be produced by the ion source at any of a variety of rates. In some cases, the ions or ion clusters may be advantageously produced at a relatively high rate. In some embodiments, the ions or ion clusters are produced at a rate of greater than or equal to 1 (ion or ion cluster) per microsecond, greater than or equal to 5 per microsecond, greater than or equal to 10 per microsecond, greater than or equal to 25 per microsecond, greater than or equal to 50 per microsecond, or greater than or equal to 75 per microsecond. In some embodiments, the ions or ion clusters are produced at a rate of less than or equal to 100 (ions or ion clusters) per microsecond, less than or equal to 75 per microsecond, less than or equal to 50 per microsecond, less than or equal to 25 per microsecond, less than or equal to 10 per microsecond, or less than or equal to 5 per microsecond. Combination of the above-referenced ranges are possible (e.g., greater than or equal to 1 per microsecond and less than or equal to 100 per microsecond). Other ranges are also possible.

In some cases, relatively high rates of ionization may be useful for the sequencing of polymers, such as biopolymers. In some embodiments, for example, the biopolymer is a protein, e.g., comprising a sequence of amino acids. In some embodiments, the protein may be ionized (i.e., produced) at any appropriate rates in one or more ranges described above, e.g., to produce amino acids (or fractions thereof) that can be analyzed as described herein to determine the sequence of the protein. As another example, in some embodiments, the biopolymer is a nucleic acid such as DNA, RNA, etc. In some embodiments, the nucleic acid may be ionized (i.e., produced) at any appropriate rates in one or more ranges described above, e.g., to produce nucleotides or other nucleic acid fragments that can be analyzed as described herein to determine the sequence of the nucleic acid. For example, the amino acid may be ionized at a rate of at least 1 base per microsecond (at least bases per microsecond, at least 100 bases per microseconds, etc.).

After exiting the ion source, in some aspects, the ions or ion clusters are emitted directly into an environment having a relatively low pressure (e.g., a vacuum chamber). The environment may have any of a variety of pressures or configurations such as those described in additional detail below. For instance, in some cases, the environment may be an environment having a pressure of no more than 100 mPa (e.g., no more than 10 mPa, no more than 1 mPa, no more than 0.1 mPa, etc.). In some embodiments, the ions or ion clusters from the fluid are passed directly into a vacuum environment, e.g., from the ion source. In addition, it should be understood that the vacuum environment need not be a perfect vacuum.

In some aspects, the emitted ions or ion clusters may pass through a mass filter, such as is described herein, for example, contained within a relatively low pressure environment. In addition, in some embodiments, prior to being passed through the mass filter, the emitted ions may optionally be passed through ionic optics, as described below.

In some embodiments, the mass filter is a magnetic mass filter. In some embodiments, the magnetic mass filter separates ions or ion clusters by their mass-to-charge ratios by applying a magnetic field. The magnetic mass filter may be capable of directing (e.g., bending) incident ions or ion clusters toward various directions (e.g., angles) according to their mass-to-charge ratios. For example, as shown in FIG. 3, incident ions and ion clusters having different mass-to-charge ratios may be directed or bent toward different directions under a magnetic field generated by the magnetic mass filter. In addition, in some embodiments, a relatively large percentage of the emitted ions or ion clusters may pass through the magnetic mass filter. For instance, at least 50% (e.g., at least 70%, at least 80%, at least 90%, at least 95%, or all) of the emitted ions or ion clusters may be passed through the magnetic mass filter. Systems and methods of determining a relatively high percentage of ions that are produced by the ion source are discussed in more detail herein.

A variety of mass filters may be used. Non-limiting examples of mass filters include, but are not limited to, quadrupole mass filters, magnetic sector mass filters, etc.

For example, as noted above, in some embodiments, the mass filter may include a magnetic mass filter. In some embodiments, the magnetic mass filter comprises a magnet and a yoke associated with (e.g., houses) the magnet. The magnetic mass filter may comprise magnets formed from any of a variety of magnetic materials, including, but not limited to rare earth elements, magnetic metallic elements, magnetic composites (e.g., ferrite), etc. In one set of embodiment, the magnet is permanent magnet comprising neodymium. In one set of embodiments, the yoke comprises iron.

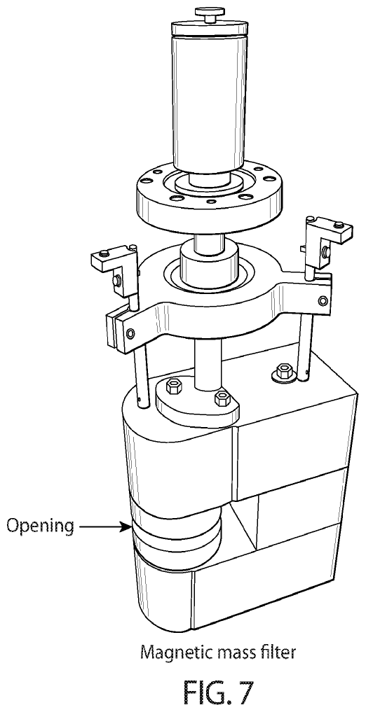

In some embodiments, a magnetic field passes through an opening of the magnetic mass filter, through which incident ions and ion clusters are passed through and which can be deflected to various degrees by the magnetic field created by the magnetic mass filter. For example, as show in FIG. 7, a magnetic mass filter has a center opening within which a magnetic field is present in the axial direction. As the ions and ion clusters travel through the center opening, the ion or ion clusters are deflected by different degrees by the magnetic field, e.g., according to their masses or mass-to charge ratios.

The opening in the magnetic mass filter may have any of a variety of dimensions and shapes. In some cases, the opening may have a shape that is cylindrical, square, rectangular, etc. The opening may have a first cross-sectional dimension (e.g., a diameter, a width, a length) and second cross-sectional dimension (e.g., a height). In one set of embodiments, the opening has a first cross-sectional dimension (e.g., diameter) that is greater than a second cross-sectional dimension (e.g., height). In some embodiments, the opening may have a first dimension (e.g., diameter) of at least 4 cm (e.g., at least 5 cm, at least 6 cm, at least 8 cm, at least 10 cm, etc.). In some embodiments, the opening may have a second dimension (e.g., height) of at least 1 cm (e.g., at least 2 cm, at least 3 cm, at least 4 cm, at least 5 cm, etc.). In some embodiments, the opening may have ratio of first dimension to second dimension of at least 2 (e.g., at least 3, at least 4, at least 5, at least 6, at least 8, at least 10, etc.).

The magnetic mass filter may generate magnetic field that has any of a variety of field strengths. In some embodiments, the magnetic field strength may be at least about 0.1 T, at least 0.2 T, at least 0.3 T, at least about 0.5 T, at least about 0.7 T, at least about 1 T, at least about 5 T, etc.

In some embodiments, optionally, ions and ion clusters may pass through an ion bender, after leaving the mass filter. The ion bender may be configured to deflect the ions and ion clusters leaving the mass filter to a detector. For instance, as a non-limiting example, ions or ion clusters are passed from an ion bender to a detector. In some embodiments, the detector can be used to determine the ions or ion clusters.

In some aspects, the ions or ion clusters passing through the mass filter (e.g., magnetic mass filter) may be directed to one or more detectors, e.g., an array of detectors (e.g., single-ion detectors) such as those described herein. For instance, as shown in FIG. 3, the magnetic mass filter directs and bends ions or ion clusters toward the array of detectors.

In some embodiments, an ion or ion cluster having a particular mass or mass-to-charge ratio may be directed toward a corresponding detector within the array of detector, for example, based on the amount of deflection that occurs as the ion or ion cluster passes through the mass filter. Ions or ion clusters with greater amounts of charge may be deflected to a greater degree than those with lesser amounts of charge. Thus, one or more detectors can be positioned to receive ions or ion clusters with different amounts of deflection, which may then be used to determine the mass or mass to charge ratio of the incident ions or ion clusters. Thus, for example, the array of detectors may be positioned to determine the mass or mass-to-weight ratio of the ions or ions clusters, based on the locations of impact of the various ions or ion clusters on various detectors within the array. In addition, in some cases, the array of detectors may include detectors that are capable of detecting the time of arrival of the respective ions or ion clusters.

In some embodiments, one or more detectors may be positioned further downstream of the mass filter. The detector may include any suitable detector able to detect ions or ion clusters. If more than one detector is present, the detectors may each independently be the same or different. Examples of specific detectors include, but are not limited to, Faraday cups, electron multipliers, dynodes, charge coupled devices (CCDs), CMOS sensors, and phosphor screens, etc.

As mentioned above, in some embodiments, a mass spectrometer comprises one or an array of detectors. In some embodiments, the array of detectors comprises a channel electron multiplier (e.g., a Channeltron® detector) and/or a dynode. Additional non-limiting examples of detects include imaging detectors such as a micro-channel plate (MCP) array, CCD, and/or CMOS sensor. More than one of these and/or other detectors may be present within an array.

The array of detectors may include any number of detectors for determining ions and/or ion clusters. For example, in some embodiments, the array of detectors may include at least 2, at least 3, at least 5, at least 10, at least 20, at least 25, at least 30, at least 40, at least 50, etc., detectors. The detectors may be positioned to receive ions or ion clusters deflected by passing through the mass filter (for example, a magnetic mass filter). For instance, the ions or ion clusters may be deflected at various angles, and the detectors forming the array positioned to receive such ions or ion clusters that are expected to be deflected by different angles. Thus, any appropriate numbers of detectors may be used to determine the mass and/or the mass-to-charge ratio of individual emitted ions or ion clusters. In some embodiments, the number of detectors may be associated with the number of base components (e.g., monomer such amino acids, nucleotides) that are present in the biopolymer.

In some aspects, systems such as those described herein may allow for mass spectrometers having a relatively high ion transmission efficiency, e.g., where ions produced at the ion source are determined using one or more detectors, e.g., within a detector array. Without wishing to be bound by any theory, it is believed that, due to the lack of air molecules within the mass spectrometer (e.g., because of the relatively low pressure environment), and/or the use of mass filters that do not lose ions or ion clusters (e.g., as would be the case with mass filters that sweeps through a narrow mass transmission window), many or even most of the ions that are produced by the ion source can be efficiently directed (e.g., using ion optics, magnetic mass filters, etc.) at the detectors, thereby resulting in surprisingly low rates of loss. Accordingly, in some embodiments, a mass spectrometer such as described herein may comprise an overall ion transmission efficiency (e.g., ratio of ions and ion clusters detected to the ions and ion clusters exiting from the fluid at the opening of the capillary) of greater than 0.01, and in some cases, at transmissions of at least 0.02, at least 0.03, at least 0.05, at least 0.1, at least 0.15, at least 0.2, at least 0.3, at least 0.4, at least 0.5, at least 0.6, at least 0.7, at least 0.75, at least 0.8, etc. In some cases, the overall ion transmission is no more than 1, no more than 0.9, no more than 0.8, no more than 0.75, no more than 0.7, no more than 0.6, no more than 0.5, no more than 0.4, no more than 0.3, no more than 0.2, no more than 0.15, no more than 0.1, no more than 0.05, or no more than 0.02. Combinations of the above-referenced ranges are possible (e.g., at least 0.02 and no more than 0.9, or at least 0.1 and no more than 0.8). Other ranges are also possible. The overall ion transmission may be measured by measuring a ratio of the detected current at the detector to the emitted current from the ion source.

Accordingly, based on such relatively high ion transmission efficiencies, in some aspects, certain systems and methods of determining molecular concentrations are described herein. The molecules may be any that can be ionized using an ion source, e.g., as discussed herein. In one set of embodiments, the molecules are dissolved in a fluid. As mentioned, the molecules may be ionized from a fluid as ions or ion clusters, after which the ions or ion clusters may be passed through a magnetic mass filter and directed to an array of detectors described herein. Examples of such molecules include, but are not limited to monomers or biomonomers (amino acids, nucleotides, etc.) such as those described herein. In addition, it should be understood that a molecule detected in such a fashion may be ionized to form more than one ion or ion cluster, e.g., such that the concentration of ions or ion clusters can be used to determine the concentration of the starting molecule.

In addition, in one set of embodiments, a fluid may comprise one or more types of molecules. In some embodiments, by determining the identities and/or the time of arrival of the one or more types of molecules, the relative amounts (e.g., concentration) of the one or more types of molecules may also be determined.

In some aspects, the ions or ions clusters arriving at the detector may be detected at a relatively high time resolution. As noted above, the time when ions or ion clusters produced at the ion source may be determined at relatively high resolutions, and such ions or ion clusters may be directed at an array of detectors, e.g., as discussed herein, without substantial impediments, such as air molecules, narrow mass transmission windows, or the like that could make it difficult to determine the timing and/or pathways of ions or ion clusters moving from the ion source to the detectors. Accordingly, in some cases, such ions and/or ion clusters may be determined, for example, at time resolution of better than 100 nanoseconds (e.g., better than 75 nanoseconds, better than 50 nanoseconds, better than 25 nanoseconds, better than 10 nanoseconds, etc.). In one set of embodiments, the time resolution may be determined between two (or more) ions or ion clusters impacting different detectors in the array.

Thus, in some embodiments, a method of determining a duration is described herein. For instance, a duration between the time the ions or ion clusters leave the ion source and the time the ions or ion clusters arrives at the detector may be determined. In some embodiments, by monitoring the time of arrival of the ions or ion clusters at the detector and the time of ion emission from the ion source, the duration may be determined. In addition, in some embodiments, the detector has an ion detection rate that is greater than or equal to the rate at which the ions or ion clusters are produced (i.e., emitted) by an ion source. In some cases, the detector may be capable of detecting each of the emitted ions or ion clusters that reaches the detector.

In some embodiments, the duration between the time the ions or ion clusters leave the ion source and the time the ions or ion clusters arrives at the detector is greater than or equal to 10 microseconds, greater than or equal to 25 microseconds, greater than or equal to 50 microseconds, greater than or equal to 75 microseconds, and greater than or equal to 100 microseconds. In some embodiments, less than or equal to 100 microseconds, less than or equal to 75 microseconds, less than or equal to 50 microseconds, less than or equal to 25 microseconds, less than or equal to 10 microseconds. Combination of the above-referenced ranges are possible (e.g., greater than or equal to 10 microseconds and less than or equal to 100 microseconds). Other ranges are also possible.

The duration between the time the ions or ion clusters leave the ion source and the time the ions or ion clusters arrives at the detector may be determined at a relatively high time resolution. For examples, the duration may be determined at a time resolution of better than 1 microsecond, better than 500 nanoseconds, better than 250 nanoseconds, better than 100 nanoseconds, better than 50 nanoseconds, better than 10 nanoseconds, better than 5 nanoseconds, etc.).

Certain aspects are directed to sequencing a polymer, such as a biopolymer, using an instrument comprising the ion source, for example, a mass spectrometer such as described herein. For instance, in some embodiments, a polymer may be the species of interest. The species of interest may be a biopolymer, e.g., a protein or peptide (comprising amino acids), or a nucleic acid sequence (e.g., DNA, RNA, etc.). Other types of biopolymers, such as carbohydrates or polysaccharides, may also be used as a species of interest in some cases. In addition, it should be understood that other types of polymers may also be sequenced in some cases, e.g., artificial or synthetic polymers. Furthermore, analogously, the structures of species of interest that are not polymers may also be determined.

In some cases, for example, the structure, sequence, and/or identity of the species of interest (e.g., a polymer) can be determined by determining the ionized fragments using a detector. For example, the sequence of the species of interest can be detected by monitoring the time of arrival of individual ionized fragments (e.g., ions or ion clusters) at the detector, e.g., that are produced by ionizing the polymer as discussed above, and producing ions or ion clusters. Without wishing to be bound by any theory, it is believed that a species of interest, such as a polymer, may be ionized in substantially linear fashion, e.g., due to the size of the opening of the capillary, and the ions or ion clusters that are produced may then be determined by a detector as discussed herein, e.g., in the order in which the ions or ion clusters are produced from the species of interest. In some embodiments, the capillary comprises a carbon nanotube or a boron nitride nanotube, where the cross-sectional dimension (e.g., inner diameter) of the nanotubes is small enough, e.g., 1 nm to 2 nm, such that a polymer molecule may ionize in a sequential order that reflects the primary structure of the polymer. Of course, larger diameters, or other materials, are also possible in other embodiments, e.g., as discussed herein. It should be noted that in some cases, e.g., when the ions or ion clusters are passed into reduced pressure environments, the detector may be able to determine such ordering at relatively high fidelity, for example due to the relative lack of collisions with gas molecules as the ions or ion clusters pass through to the detector. Accordingly, based on the order at which ions or ion clusters are determined, the structure or sequence of the species of interest can be determined.

In some embodiments, a method of sequencing a species of interest is described herein. In one set of embodiments, the sequence of a species of interest (e.g., biopolymer) is determined by determining the ions or ion clusters with an array of detectors. As noted above, molecules in a species of interest (e.g., a biopolymer) may be ionized using an ion source to produce a sequence of ions or ion clusters (e.g., ions with solvent molecules). In some cases, the ion source may produce predominantly single ions with little, if any, of ion clusters.

In some embodiments, the sequence of the ions or ion clusters (e.g., single ions) emitted from the ion source may substantially retain the sequence of the biopolymer prior to ionization. For instance, at least 50% (at least 60%, at least 70%, at least 80%, at least 90%, at least 95%, at least 99%, etc.) of the ions or ion clusters are emitted in sequence. As the sequence of ions or ion clusters passes through a mass filter, such as a magnetic mass filter, the ions or ion clusters may be directed to an array of detectors. Accordingly, in certain embodiments, the array of detectors may be used to determine the mass-to-charge ratio of the ions or ion clusters and/or the time of arrival (e.g., time at detection) of the ions or ion clusters.

Advantageously, the time of arrival of the ion or ion clusters may provide information about the sequence of ions or ion clusters at detection, in accordance with some embodiments. In some cases, a substantial fraction of the ions or ion clusters arrive at the array of detectors (e.g., single-ion detectors) arrive in sequence, e.g., the same sequence of the emitted ions or ion clusters from the ion source. For instance, at least 50% (at least 60%, at least 70%, at least 80%, at least 90%, at least 95%, at least 99%, etc.) of the ions or ion clusters arriving at the array of detectors arrive in sequence. Accordingly, the array of detectors may be used in some embodiments to determine the identity of the ions or ion clusters and/or the sequence of the ions or ion clusters. In some instances, the sequence of the species of interest (e.g., a biopolymer) can be determined based on the sequence of the ions or ion clusters.

Some aspects are directed to a mass spectrometer comprising an ion source as described herein. In some cases, the mass spectrometer may include, besides an ion source such as described herein, components such as vacuum chambers (e.g., able to produce any of the reduced pressures described herein), ion optics (e.g., one or more lenses such as Einzel lenses, etc.), mass filters (e.g., quadrupole mass filters, magnetic sector mass filters, etc.), detectors, ion benders, ion traps, or the like. These and other components are discussed in more detail herein.

For example, in one set of embodiments, a mass spectrometer or other device as discussed herein may comprise an ion source having a capillary as disclosed herein. The device may also have an electrode proximate the capillary. Certain embodiments, for instance, are directed to an ion source comprising a capillary defining an opening and an electrode posited proximate the opening. The capillary may have an opening at an end or a tip of the capillary. The opening may have any of a variety of cross-sectional dimensions, and may be of any shape, e.g., circular, elliptical, square, etc. In some embodiments, the opening comprises a cross-sectional dimension of less than 150 nm, less than 130 nm, 125 nm, less than 120 nm, less than 110 nm, less than 100 nm, less than 90 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, less than 10 nm, less than 5 nm, less than 2 nm, etc. In addition, the opening, in some cases, may have a cross-sectional dimension of at least 1 nm, at least 5 nm, at least 10 nm, at least 15 nm, at least 20 nm, at least 25 nm, at least 30 nm, at least 40 nm, at least 50 nm, at least 60 nm, at least 70 nm, at least 80 nm, at least 90 nm, etc. Combinations of these are also possible; for example, the opening may have a cross-sectional dimension of between 50 nm and 100 nm. While the above embodiment describes a capillary having an opening at the end or the tip of the capillary, it should be understood that not all embodiments described herein are so limiting, and in certain embodiments, the capillary may additionally or alternatively have a plurality of openings along the side of the capillary. In addition, in some cases, a device may have one or more apertures or openings, e.g., in a channel or other structure. Thus, an opening need not be the opening of a capillary.

In some embodiments, the capillary is tapered at the opening. For instance, the capillary may have a constant tapering, e.g., such that the tip of the capillary is cone-shaped. Any suitable angle may be present. For example, the angle may be less than 15 degrees, less than 10 degrees, less than 9 degrees, less than 8 degrees, less than 7 degrees, less than 6 degrees, less than 5 degrees, less than 4 degrees, less than 3 degrees, less than 2 degrees, or less than 1 degree (where 0 degrees indicates no taper, i.e., the capillary is cylindrical. In addition, in some cases, the angle of the taper may be at least 1 degree, at least 3 degrees, at least 5 degrees, etc., in certain cases. Combinations of these ranges are also possible, e.g., the tapering may be between 1 degree and 5 degrees.

In certain embodiments where the capillary is tapered at the opening, a laser pulling technique can be used to fabricate the tapered opening. It should be understood that techniques other than a laser-pulling technique could also be used to produce capillaries with tapered openings. It should also be understood that, although the capillary discusses herein has a tapered opening, in other examples, the opening of the capillary could be non-tapered.

The capillary of the ion source comprises quartz in certain embodiments. Additional examples of materials that can be used to fabricate the capillary include, but are not limited to, glass (e.g., borosilicate glass), a plastic, a metal, a ceramic, a semiconductor, a carbon nanotube, a boron nitride nanotube, etc.

In some embodiments, the capillary has a relatively high aspect ratio, e.g., a ratio of the length of the capillary to the cross-sectional dimension (e.g., diameter) of the capillary's opening. For example, the capillary may have an aspect ratio that is greater than 10,000. However, it should be understood that the aspect ratio is not so limited. For instance, in some examples, the aspect ratio of the capillary length to the opening's cross-sectional dimension may be greater than 10, greater than 100, greater than 1,000, greater than 10,000, greater than 100,000, or greater than 1,000,000.

The capillary may have a circular or a non-circular cross-section (e.g., square). In addition, in some embodiments, the capillary may have a relatively small cross-section, e.g., diameter. For instance, the cross-sectional dimension of the capillary may be less 200 nm, less than 150 nm, less than 100 nm, less than 75 nm, less than 60 nm, less than 50 nm.

Certain embodiments of the ion source also comprise an electrode positioned proximate the capillary, e.g., the opening of the capillary. The electrode may be used to apply an electric field (for example, as described below) to a fluid within the capillary, e.g., to be applied to the meniscus. In some cases, the fluid within the capillary may be in contact with a counterelectrode, e.g., such that a voltage difference between the electrode proximate the opening of the capillary and the counterelectrode within the capillary is able to generate an electric field to the fluid. In some embodiments, the electrode may be positioned so as to cause an electric field maximum proximate the opening of the capillary. For example, in some embodiments, the electrode may be positioned within 50 mm, within 40 mm, within 30 mm, within 20 mm, within 15 mm, within 10 mm, within 5 mm, within 3 mm, within 2 mm, within 1 mm, etc., of the opening of the capillary.

The electrode, in some embodiments, may be positioned around the capillary, or may be positioned in front of the capillary, e.g., in front of the opening of the capillary, or in a downstream direction.

The electrode may have any suitable shape. In some cases, the electrode is circular or circularly symmetric, or is symmetrically positioned with respect to the capillary. However, other shapes or arrangements are also possible.

In some embodiments, the electrode defines an opening (e.g., an aperture). Thus, the electrode may be annular in some cases. The electrode may be positioned such that ions or ion clusters escaping the fluid in the capillary pass through the center opening of the electrode. The center opening of the electrode can be of any shape, including, but not limited to, a circular shape that can be positioned annularly around the opening of the capillary. The opening may also be non-circular in some cases. In some embodiments, the opening of the electrode is positioned coaxially to the opening of the capillary. That is, the opening can be aligned, in certain embodiments, to the opening of the capillary, e.g., such that an imaginary line passing through the center of a cross-section of the capillary passes through the center opening of the electrode. This may facilitate the application of an electric field to the fluid in the capillary, e.g., to cause ions or ion clusters to exit the fluid, as discussed herein.

For example, in some embodiments, the electrode has a center opening with cross-sectional dimension (e.g., inner diameter) greater than the cross-sectional dimension of the opening of the capillary, e.g., at the end or tip of the capillary. For instance, in accordance to certain embodiments, the electrode has a center opening with a cross-sectional dimension (e.g., inner diameter) at least 5 times greater than the cross-sectional dimension of a capillary's opening. However, it should be understood that the ratio of the cross-sectional dimensions of the electrode's center opening to the capillary's opening is not limited. For instance, in some examples, the cross-sectional dimension of the center opening of the electrode could be at least 2 times, at least 3 times, at least 5 times, at least 10 times, at least 20 times, at least 30 times, at least 50 times, at least 75 times or at least 100 times larger than the cross-sectional dimension of the capillary's opening. In certain cases, the opening of the electrode may have a cross-sectional dimension of less than 10 cm, less than 5 cm, less than 3 cm, less than 1 cm, less than 5 mm, less than 3 mm, less than 1 mm, etc. In addition, in some embodiments, a front side of the electrode is positioned in front of the opening of the capillary.

In addition, the electrode itself can be of any shape (e.g., circular or non-circular). The electrode may have the same or a different shape than its opening (if present). The electrode may have any suitable cross-sectional dimension. For example, the electrode may have a cross-sectional dimension of less than 10 cm, less than 5 cm, less than 3 cm, less than 1 cm, less than 5 mm, less than 3 mm, less than 1 mm, etc.

In some embodiments, the electrode comprises steel. Other examples include copper, graphite, silver, aluminum, gold, electrically conducting ceramics, or the like.

Thus, certain embodiments are directed to an electrode able to generate an electric field. In some cases, as noted, the electrode may be positioned to create an electric field maximum proximate the opening of the capillary. In some embodiments, a fluid is housed in the capillary such that when an electric field is applied by the electrode proximate the opening of the capillary, molecules within the fluid can ionize and exit from the opening of the capillary, e.g., as ions or ion clusters such as discussed herein. In some cases, for example, the electrode and the capillary (e.g., the interior of the capillary) may be connectable to a voltage source, e.g., as discussed herein.

Thus, in certain embodiments, the voltage source, in conjunction with the electrodes, may be used to produce an electric field to cause ions or ion clusters to exit a fluid in the capillary, e.g., as discussed herein. In some embodiments, a voltage is applied to generate an electric field at least sufficient to ionize molecules within the fluid at the opening of the capillary, e.g., to produce ions or ion clusters. For instances, in certain embodiments, a voltage in the range of 80 V to 400 V could be used to generate an electric field. In some cases, the voltage may be at least 40 V, at least 60 V, at least 80 V, at least 100 V, at least 120 V, at least 140 V, at least 160 V, at least 180 V, at least 200 V, at least 220 V, at least 240 V, at least 260 V, at least 280 V, at least 300 V, at least 320 V, at least 340 V, at least 360 V, at least 380 V, at least 400 V, at least 450 V, at least 500 V, at least 600 V, etc. In addition, in some cases, the voltage may be no more than 600 V, no more than 500 V, no more than 450 V, no more than 400 V, no more than 380 V, no more than 360 V, no more than 340 V, no more than 320 V, no more than 300 V, no more than 280 V, no more than 260 V, no more than 240 V, no more than 220 V, no more than 200 V, no more than 180 V, no more than 160 V, no more than 140 V, no more than 120 V, no more than 100 V, no more than 80 V, no more than 60 V, etc. In some cases, combinations of these voltages are possible. For instance, the voltage may be applied between 80 V and 360 V, etc. The voltage may be applied as a constant voltage, or a varying or periodic voltage in certain cases.

As mentioned, a voltage may be applied to create an electric field maximum proximate the opening of the capillary, or the fluid within the capillary (e.g., at the meniscus at the opening). For example, a voltage may be applied to create an electric field maximum of at least 0.5 V/nm, at least 0.7 V/nm, at least 1 V/nm, at least 1.1 V/nm, at least 1.3 V/nm, at least 1.5 V/nm, at least 2 V/nm, at least 2.5 V/nm, at least 3 V/nm, at least 3.5 V/nm, at least 4 V/nm, etc. In certain embodiments, the electric field maximum may be no more than 5 V/nm, no more than 4.5 V/nm, no more than 4 V/nm, no more than 3.5 V/nm, no more than 3 V/nm, no more than 2.5 V/nm, no more than 2 V/nm, no more than 1.5 V/nm, no more than 1 V/nm. Combinations of these ranges are also possible in some embodiments; for example, the electric field may be between 1.5 V/nm and 3.0 V/nm, between 1.5 V/nm and 4.0 V/nm, etc.

Without wishing to be bound by any theory, it is believed that in certain embodiments, when the electric field is applied, fluid within the capillary forms a charged meniscus and species exit the charged meniscus, e.g., as ions or ion clusters. In some cases, the opening of the capillary may be sized such that at least 10% of the exiting species exit via ion evaporation, e.g., as ions or ion clusters. In some cases, at least 20%, at least 30%, at least 40%, at least 50%, at least 60%, at least 70%, at least 80%, at least 90%, at least 95%, etc. of the exiting species exit via ion evaporation.

As described previously, according to certain embodiments, a charged fluid meniscus in the shape of a cone can be generated at the opening of a capillary under an electric field. In some embodiments, the conical fluid meniscus acts as a point source to allow species to exit as ions or ion clusters.

The fluid meniscus could produce exiting species via mechanisms such as charged droplets via electrospray ionization, and/or ions and ion clusters via ion evaporation. However, in the case of electrospray ionization, the exiting species exiting from the liquid meniscus would exit as charged droplets of fluid containing the exiting species, which would require the presence of a background gas to further break down the droplets into individual ions, typically via a Coulomb fission process. Ion evaporation, on the other hand, describes a process where a molecule is directly ionized into ions (e.g., bare ions) or ion clusters (e.g., ions with solvent molecules), instead of charged droplets. An ion cluster may contain a single ion and a plurality of solvent molecules, usually a relatively small number. For example, the ion clusters may contain no more than 10, no more than 9, no more than 8, no more than 7, no more than 6, no more than 5, no more than 4, no more than 3, no more than 2, or no more than 1 solvent molecule.

Thus, for example, in some embodiments, the opening of the capillary is sized (e.g., the cross-sectional dimension of the opening is less than 100 nm) such that the formation of charged droplets can be avoided, and such that at least 50% of the exiting species directly ionize as ions or ion clusters from conical fluid meniscus at the opening of the capillary.

As mentioned, in some embodiments, a capillary having a relatively small opening (e.g., a cross-sectional dimension of less than 100 nm) may be associated with the production of a relatively small number of solvent molecules in an ion cluster, e.g., as described above. In some embodiments, the opening of the capillary may be sized (e.g., less than 100 nm) such that the plurality of solvent molecules comprises less than or equal a certain number of solvent molecules, e.g., such that on average, the ion clusters produced by the ion source contain less than or equal to 7, 6, 5, 4, 3, or 2 solvent molecules. In some embodiments, a substantial number (e.g., greater than or equal to 50%, greater than or equal to 60%, greater than or equal to 70%, greater than or equal to 80%, greater than or equal to 80%, greater than or equal to 90%, greater than or equal to 95%, greater than or equal to 99%, or all) of the ion clusters contains one or two solvent molecules.

In addition, as discussed, certain embodiments are directed to methods of ionizing a fluid using an ion source, e.g., to produce single ions or ion clusters. Certain embodiments comprise passing a fluid into a capillary defining an opening having a cross-sectional dimension less than 100 nm, or other configurations such as those discussed herein.

In some embodiments, the fluid comprises a sample and a solvent. The sample may include any species of interest that can be ionized from the opening of the capillary. For instance, in accordance with certain embodiments, a species of interest comprises a biopolymer (e.g., nucleic acids such as DNA or RNA, peptides or proteins, etc.). Other examples include other types of polymers, e.g., nylon, polyethylene, etc., or other species of interest that are not necessarily polymers, e.g., biomolecules. Non-limiting examples of biomolecules may include monomers such as amino acids, nucleotides, etc. In some cases, the species of interest is unknown, and it is desired that the structure of the species be at least partially determined, e.g., by ionizing the species and detecting the ion fragments, such as in mass spectroscopy or other related techniques.

In some embodiment, the solvent may be any liquid that can be used to dissolve the sample or the species of interest. For instance, in accordance with certain embodiments, the solvent comprises water. However, the solvent is not limited to water. In some cases, the solvent may be an aqueous solution, e.g., having any of a variety of salt concentrations. In some embodiments, an aqueous solution may have a salt concentration of greater than or equal to 10 mM, greater than or equal to 20 mM, greater than or equal to 30 mM, greater than or equal to 50 mM, greater than or equal to 100 mM, greater than or equal to 150 mM, greater than or equal to 200 mM, greater than or equal to 300 mM, greater than or equal to 400 mM, greater than or equal to 500 mM, greater than or equal to 750 mM, greater than or equal to 1 M, greater than or equal to 2 M, greater than or equal to 5 M, or greater than or equal to 7.5 M. In some embodiments, an aqueous solution may have a salt concentration of less than or equal to 10 M, less than or equal to 7.5 M, less than or equal to 5 M, less than or equal to 2 M, less than or equal to 1 M, less than or equal to 750 mM, less than or equal to 500 mM, less than or equal to 400 mM, less than or equal to 200 mM, less than or equal to 150 mM, less than or equal to 100 mM, less than or equal to 50 mM, less than or equal to 30 mM, less than or equal to 20 mM, less than or equal to 10 mM, etc. Combination of the above-referenced ranges are possible (e.g., greater than or equal to 100 mM and less than or equal to 10 M, or greater than or equal to 150 mM and less than or equal to 1 M).

Additional examples of solvents that can be used include, but are not limited to, formamide, alcohols (e.g., ethanol, isopropanol, etc.), organic solvents (e.g., toluene, acetonitrile, acetone, hexane, etc.), ionic liquids, inorganic solvents (e.g., ammonia, sulfuryl chloride fluoride, liquid acids and bases, etc.). Combinations of any of these and/or other solvents are also possible in certain cases.

In addition, in some embodiments, the fluid comprises a solvent (e.g., water) having a relatively high volatility, e.g., to facilitate the production of ions or ion clusters. For instance, water, with its boiling point of 100° C., can be considered to be volatile in some cases. In some embodiments, liquids with boiling points close to room temperature could be used to facilitate the production of ions or ion clusters. In some embodiments, a solvent that could be used to facilitate the production of ions or ion clusters may have a boiling point of less than or equal to 100° C., less than or equal to 80° C., less than or equal to 60° C., less than or equal to 40° C., less than or equal 20° C., etc. In addition, the solvent may have a boiling point greater than or equal to 10° C., greater than or equal to 30° C., greater than or equal to 50° C. greater than or equal to 70° C., greater than or equal to 90° C., etc. Combination of these are also possible; for example, the solvent may have a boiling point of between 50° C. and 100° C. Additional examples of solvents having a relatively high volatility include, but are not limited to, acetone, isopropanol, hexane, etc.

In some embodiments, the temperature of the capillary (in addition to the type of fluid it contains) may be varied to control the number of solvent molecules in a resultant ion cluster. In some embodiments, the temperature of the capillary is set such that the plurality of solvent molecules comprises less than or equal a certain number of solvent molecules, e.g., such that on average, the ion clusters produced by the ion source contain less than or equal to 7, 6, 5, 4, 3, or 2 solvent molecules. In some embodiments, the temperature is at least 20° C., at least 30° C., at least 40° C., at least 50° C., at least 60° C., or at least 70° C. In some embodiments, the temperature is no more than 80° C., no more than 70° C., no more than 60° C., no more than 50° C., no more than 40° C., no more than 30° C. Combination of the above-referenced ranges are possible (e.g., greater than or equal to 20° C. and less than or equal to 80° C.). In some cases, the temperature of the capillary is controlled by a resistive heater, by a Peltier junction, by an infrared heater, etc.

In some embodiments, an appropriate range of electric field and an appropriate range of sizes of the capillary opening can be selected to cause at least some of the molecules to exit as ions or ion clusters, e.g., as discussed herein.

In certain embodiments, an ion source as described herein may be used with a liquid-chromatography mass spectrometry system. For instance, a liquid chromatograph can be coupled with an ion source to separate peptides or other molecules before ionizing and delivering them into a mass spectrometer. In some cases, the mass spectrometer may be used to perform a single or tandem (MS/MS) analysis to identify the ionized peptides or molecules, as in a proteomics experiment. Advantageously, the use of the ion source (having a capillary with a nanosized opening and/or tip) described herein to deliver ions directly into a low pressure environment may improve the sensitivity of the instrument, the ion transmission efficiency in such a system, and remove the need for multiple pumping stages.

In other embodiments, an ion source as described herein may be used as both a nanopipette and an ion source. For instance, a capillary (e.g., a pulled quartz capillary) described herein having a nanosized tip may be used to puncture a cell or tissue and withdraw its biomolecular contents. The capillary may then be directly inserted into a vacuum chamber and the extracted molecules may be ionized and delivered to a mass spectrometer. Such techniques may be used, for example, to sample relatively small liquid volumes, such as the contents of a single cell. For instance, such a technique may be used for single cell proteomics studies.

According to certain aspects, in addition to an ion source, various ion optics can be positioned downstream of the ion source such that the exiting molecules (e.g., ions and ion clusters) can be transported along a path downstream of the ion source in certain cases, i.e., the downstream direction is the direction in which ions or ion clusters travel. In some embodiments, the ion optics comprises one or more Einzel lenses (e.g., a first Einzel lens and a second Einzel lens). Those of ordinary skill in the art will be familiar with various ion optics used in mass spectrometry.

Certain aspects comprise passing the ionized molecules from the fluid directly into a reduced pressure or vacuum environment. Without wishing to be bound by any theory, it is noted that techniques such as electrospray ionization typically requires the presence of a background gas to further break down the droplets into individual ions, typically via a Coulomb fission process. In contrast, in accordance with certain embodiments, ions or ion clusters produced as discussed herein can be directly passed into such an environment, without requiring substantial amounts of background gas. Thus, certain techniques such as mass spectrometry may be performed using a reduced pressure or vacuum environment, without necessarily requiring the addition of a background gas.

Thus, in one set of embodiments, the capillary may be positioned to allow ions or ion clusters exiting the opening to enter a reduce pressure or vacuum environment. In some cases, the environment may be an environment having a pressure of no more than 100 mPa. In certain embodiments, the environment may have a pressure of no more than 1000 mPa, no more than 10 mPa, no more than 1 mPa, no more than 0.1 mPa, etc. In some embodiments, the ions or ion clusters from the fluid are passed directly into a vacuum environment.

It should be understood that some of the embodiments provided herein focused on passing the ionized molecules from the fluid directly into an environment having a pressure of no more than 100 mPa. However, it should be understood the pressure within the environment is not limited to 100 mPa. In some embodiments, the pressure could also be greater than or equal to 100 mPa, and less than or equal to 1 Pa.

As mentioned previously, in some embodiments, the mass spectrometer comprises a pump. The pump may be used to create a reduced pressure or vacuum environment, e.g., as discussed herein. Non-limiting examples of pumps include diffusion pumps, molecular drag pumps, turbomolecular pumps, or the like.

In some embodiments, there may be a relatively high pressure difference between the vacuum chamber and the fluid at the capillary opening. For instance, the pressure may be about 1 atmosphere at where the fluid enters in the capillary and about 100 mPa, or other reduced pressures such as those described herein, inside the vacuum chamber where the opening of the capillary is located. However, in some cases such as are described herein, the fluid meniscus at the opening of the capillary may be relatively stable despite the relatively high pressure difference, e.g., due to the surface tension of the fluid at the meniscus. For instance, the pressure difference across the fluid meniscus at the opening of the capillary may be at least 0.1 atm, at least 0.2 atm, at least 0.3 atm, at least 0.4 atm, at least 0.5 atm, at least 0.6 atm, at least 0.7 atm, at least 0.8 atm, at least 0.9 atm, at least 1 atm, etc. Furthermore, in some embodiments, the hydraulic resistance of a fluid in a capillary such as described herein (e.g., a capillary with an opening less than 100 nm) may be higher than that in an ion source employed in electrospray ionization.

In accordance with certain embodiments, the opening of the capillary is sized such that a solvent having a relatively high volatility remain unfrozen at the opening of the capillary when exposed to relatively low pressures. In some embodiments, the opening of the capillary is small enough such that a solvent of relatively high volatility remains unfrozen as the solvent enters the surrounding environment. In some embodiments, the opening of the capillary is small enough such that a fluid comprising a sample and a solvent remains unfrozen as the species of interest ionizes, such that at least some of the species of interest ionizes to form ions (e.g., single ions) or ion clusters.

U.S. Provisional Patent Application Ser. No. 63/015,407, filed Apr. 24, 2020, entitled “Nanotip Ion Sources and Methods,” by Stein, et al., is incorporated herein by reference in its entirety. In addition, International Patent Application Serial No. PCT/US2021/028954, filed Apr. 23, 2021, entitled “Nanotip Ion Sources and Methods,” by Stein, et al., is also incorporated herein by reference in its entirety. Furthermore, U.S. Provisional Patent Application Ser. No. 63/179,046, filed Apr. 23, 2021, entitled “Systems and Methods for Single-Ion Mass Spectrometry with Temporal Information,” by Stein, et al., is also incorporated herein by reference in its entirety.

The following examples are intended to illustrate certain embodiments of the present disclosure, but do not exemplify the full scope of the disclosure.

### Example 1

Disclosed in the following examples are certain systems and methods that allowed the measurement of a sample (e.g., amino acids) directly emitted into vacuum (i.e., a reduced pressure environment) via ion evaporation from the surface of a fluid, e.g., an aqueous solution in this example. The systems and methods in these examples were applicable to fluids with relatively low conductivities, e.g., equivalent to about 10 mM NaCl. In some embodiments, the methods also predominantly produced bare ions or ionic clusters with only one or two water molecules. One feature was directed to the nanoscale size (<100 nm) of the ion source. The small size of the capillary tip produced significant field enhancement and restricted the fluid flow rate such that ions from the samples (e.g., amino acids) exited directly into vacuum via ion evaporation rather than from the formation of droplets and a sequence of Coulomb fissions. The small tip opening (sometimes also called a “nanopore”) also prevented the evaporation of significant amounts of solvent into the vacuum chamber while keeping the fluids from freezing, allowed the study of samples such as amino acids in volatile solvents such as water.

This example illustrates various parts of the mass spectrometer instrument that was used to conduct experiments in accordance with one embodiment. The experiments described in the following examples were all conducted in a custom instrument called a “nanopore mass spectrometer,” illustrated schematically in FIGS. 1A-1B.

One component of the instrument used in this example is an ion source. The ion source includes a capillary with a sub-100 nm inner tip diameter and an annular electrode situated in front of the capillary tip within the system. The capillary was made of quartz in this example, but capillaries could also have been made of borosilicate glass, plastics, metals, ceramics, semiconductors, or other materials in other embodiments. The diameter of the capillary in this example tapered gradually down in size approaching the tip, such that if one approximated the shape of the tip to be a cone, the opening angle of the cone would be in the range of about 1 degree to 5 degrees. The capillary was much longer than it is wide at the tip, giving it an extremely high aspect ratio, typically above 10,000. Of course, as previously discussed, other capillary shapes and/or dimensions can be used in other embodiments.

The electrode was used in this example to induce an electric field at the opening of the tip of the capillary that was high enough to allow ions to exit directly from the meniscus of the fluid created there, at least in part by ion evaporation. The electrode was made of steel in this instrument, but it could have been made from another conducting material. The electrode in this example featured an aperture through which ions could travel, e.g., exiting from the meniscus of the fluid by ion evaporation. In this instrument, the electrode had the shape of a washer, i.e., a circular disk with a circular hole in the middle, although other shapes could have been used. The diameter of the hole in the middle was about 1 cm in this instrument, but that dimension was not critical. For example, it may be at least 10 times larger than the capillary tip diameter, or other dimensions as described herein. The outer diameter of the electrode was about 5 cm, but that dimension was also not critical. It may be larger than the inner hole diameter. The front side of the electrode in this experiment defined a plane, and the tip of the capillary was situated behind that plane at a distance in the range of about 1 mm to 5 mm, with the capillary axis aligned with the axis of the electrode.

A voltage typically in the range 80 V-400 V was applied between the fluid and the electrode to cause ions to exit the fluid. An Ag/AgCl wire in the capillary was used as the counterelectrode. The exiting beam of ions passing through the aperture in the electrode was focused using two Einzel lenses. The ions were analyzed by a quadrupole mass filter in the instrument, but a different type of mass filter, e.g., a magnetic sector, could also have been used.

### Example 2

This example illustrates a mass spectrometer capable of determining the mass-to-charge ratio (m/z) and precise moment of detection of an ion emitted at an arbitrary time from a nanopore ion source, in accordance with certain embodiments. Knowledge of the relative timing of ion detections allows determinations of ion associations, ion orderings, and ion sequences. The instrument in this example derives its ability to provide temporal information by combining a magnetic mass filter with an array of single ion detectors. This example demonstrates that such an instrument is sensitive to multiple different amino acids arriving simultaneously, and that its temporal resolution is less than a microsecond.

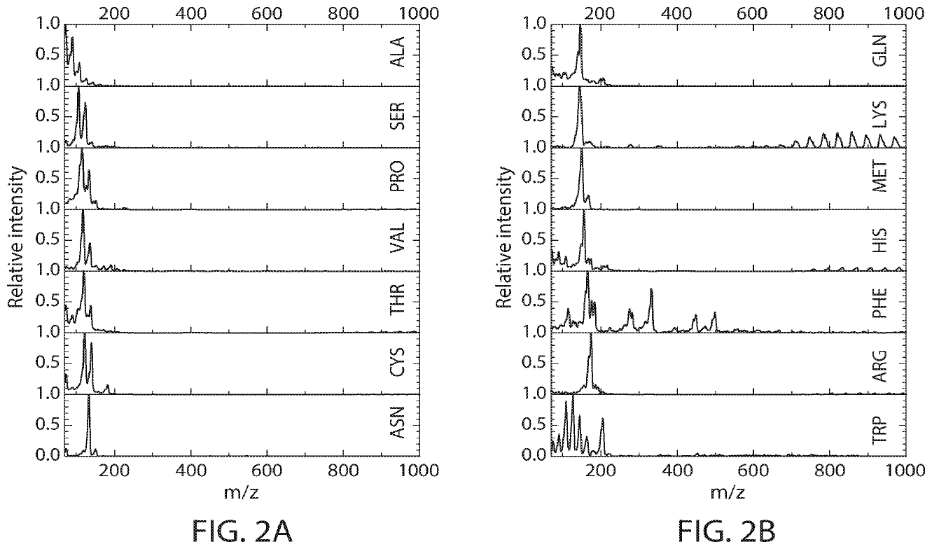

A nanopore ion source can deliver single amino acid ions directly into the high vacuum part of a mass spectrometer from solutions of formamide or water. See U.S. Pat. Apl. Ser. No. 63/015,407, filed Apr. 24, 2020, entitled “Nanotip Ion Sources and Methods,” by Stein, et al., and a PCT application filed on Apr. 23, 2021, entitled “Nanotip Ion Sources and Methods,” by Stein, et al., each incorporated herein by reference in its entirety. For example, FIG. 2 shows the mass spectra for 14 different amino acids delivered from aqueous solutions by capillary tips with diameters smaller than 100 nm. Remarkably, the amino acid ions are predominantly bare, as opposed to clustered with solvent molecules; this makes interpretation of the data relatively straightforward. It is also remarkable that these high quality mass spectra were obtained from very small ion emission currents (˜10 pA) and using very low extraction voltages (˜200 V).

In FIG. 2, the mass spectra of positive amino acid ions delivered directly into high vacuum from a nanopore ion source are shown. The amino acids were dissolved in aqueous solutions. In each case, the pH was adjusted using acetic acid to fall below the isoelectric point of the amino acid in solution.

In this example, an instrument was designed and fabricated that was capable of determining single ions. This is shown schematically in FIG. 3. The instrument combines a nanopore ion source with a magnetic mass filter and an array of single ion detectors. The magnetic mass filter was built using a neodymium magnet and an iron yoke. The mass filter created a cylindrical region about 5 cm in diameter and 1 cm in height within which there was a magnetic field oriented in the axial direction. The magnetic field measured approximately 0.6 T. Ions passed through the cylindrical region horizontally (perpendicular to the cylinder axis) and experienced a magnetic force that causes the ions to fan out according to their m/z. Arrays of single ion detectors received the fan of ions and determine the m/z of each ion by the location of impact.

The ion detector array can comprise Channeltron® detectors (e.g., electron multipliers), and dynodes. The detector array can also comprise imaging detectors such as micro-channel plate (MCP) arrays, CCDs, or CMOS sensors. Because the instrument in this example, determines the mass of an ion based where it hits the detector array, the time domain is available to establish the order in which the ions were emitted. Thus, the instrument can measure amino acid sequences by interpreting the order of detector array pulses.

### Example 3

This example describes a technology for sequencing single proteins. Approaches to single-protein sequencing based on fluorosequencing, nanopores, and tunneling spectroscopy are under development and show promise. However, only mass spectrometry (MS) has demonstrated an ability to identify amino acids with minimal degeneracy. Existing MS ion sources have low ion transfer efficiencies and scramble the ions' spatial ordering. Presented herein is an ion source comprising a glass capillary with a sub-100 nm diameter orifice that emits amino acid ions from aqueous solution directly into high vacuum. Single ions travel collision-less trajectories before striking a single ion detector. In this example, unsolvated ions of 16 different amino acids as well as glutathione and two of its post-translationally modified variants were measured. This example discusses an approach to sequencing single proteins based on MS and the nanocapillary ion source.

Mass spectrometry has been the workhorse of proteomics research for decades; its utility derives from an ability to distinguish amino acids by their mass and the availability of fragmentation techniques that can probe protein structure in tandem MS (MS/MS) measurements. Also, the development of soft ionization techniques, in particular electrospray ionization (ESI), has been crucial to transfer peptide ions into the vapor phase intact. However, ESI suffers from low ion transfer efficiency, which limits the sensitivity of mass spectrometry; millions to billions of protein copies are needed to reach the detection limits of a typical instrument.

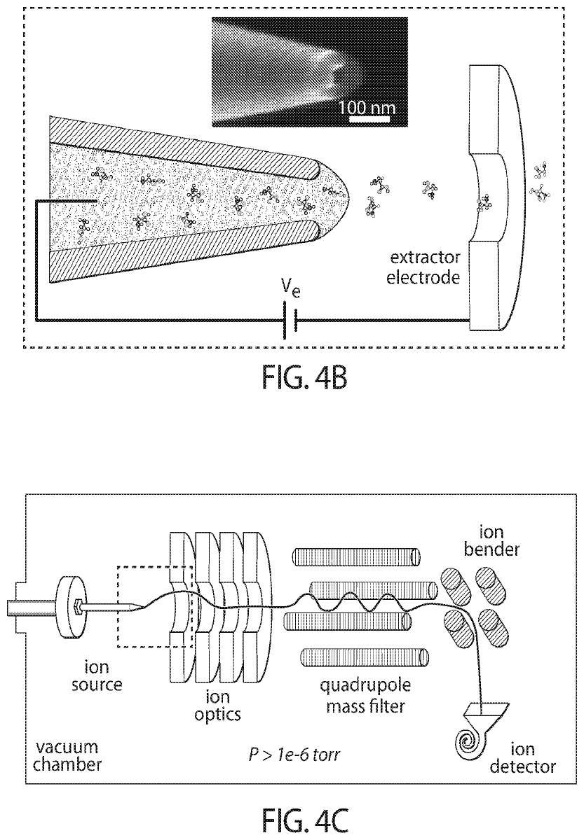

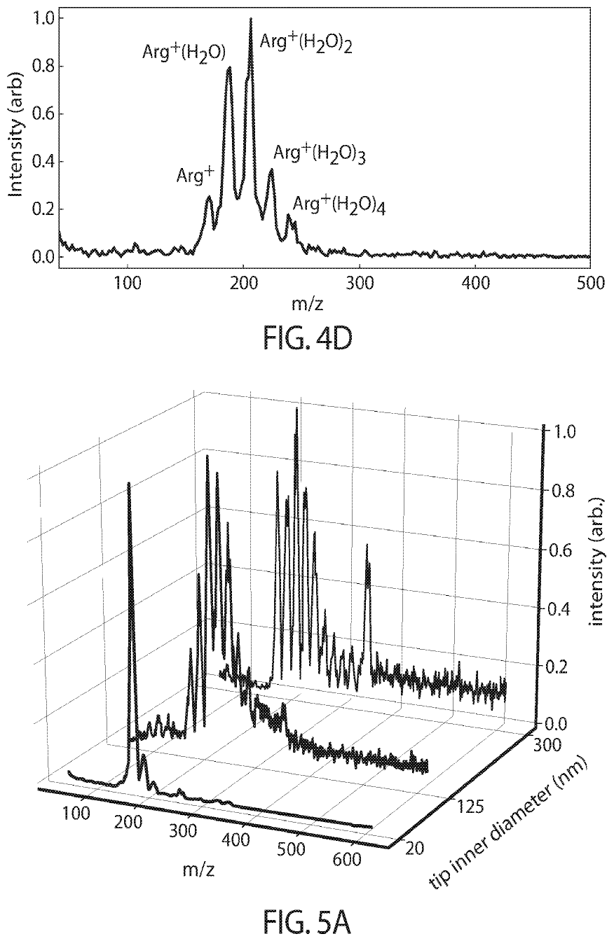

ESI delivers analyte into a mass spectrometer from a plume of charged droplets that emerge from an electrically induced liquid cone-jet at the end of a capillary, as illustrated in FIG. 4A. Each droplet, which carries large numbers of charges and analyte molecules, must undergo a string of evaporation and Coulomb explosion cycles before ions are available for analysis in the vapor phase. Only a fraction of the analyte molecules ultimately emerge as vapor phase ions, and of those that do, a large fraction collide with the walls of the transfer capillary before entering the low pressure region where the mass filter and detector are housed. ESI typically transfers just 1 in 106 ions into the mass filter. The nano-electrospray ionization (nano-ESI) technique raises the transmission efficiency to the range 0.1%-1% by using smaller capillaries (around 1 micrometer tip diameter). Hydrodynamic ion focusing is another technique that significantly increases the transmission efficiency using optimized apertures between vacuum stages. However, even if the transfer efficiency approaches unity, all of the aforementioned techniques face a separate challenge towards sequencing. The use of an atmospheric-pressure background gas to promote the desolvation of ions creates an environment where the mean free path of an amino acid is less than 50 nm, and collisions will quickly scramble the spatial ordering needed to sequence a single protein.

Described in this example is an ion source that emits amino acid ions directly into high vacuum (FIG. 4B). The heart of the ion source is a pulled quartz capillary whose tip diameter is smaller than 100 nm. The smallness of the tip can influence emission in various ways: First, the fluid flow rate may be about three orders of magnitude lower than in nano-ESI and below the minimum flow rate needed to form a stable cone-jet. In the absence of a cone-jet, charge droplets might be prevented from forming. In some cases, the surface tension of a water meniscus stretched across a nanoscale opening can support many atmospheres of pressure and maintain a stable liquid-vacuum interface. In addition, in some cases, electric fields concentrate at a sharp, conductive tip like an electrolyte-filled nanocapillary. As a result, electric fields of ˜1 V/nm can be achieved at the liquid meniscus under certain conditions. At that high field strength, ions would escape from the liquid at high rates by the process of ion evaporation.

An overview of the mass spectrometer described in this example is shown in FIG. 4C. Ions were emitted from the source by applying a voltage between a Ag/AgCl electrode inside the nanocapillary and a ring-shaped extraction electrode located about 5 mm in front of the nanocapillary tip. The ions passed through focusing ion optics, a quadrupole mass filter (Extrel), and an ion bender, before being measured by a continuous dynode single-ion detector. The background pressure inside the instrument was typically 10-6 torr, and the mean free path of an amino acid (>10 m) was more than an order of magnitude larger than the size of the instrument.

Aqueous solutions of 16 different amino acids were prepared at concentrations of 100 mM, with the exception of tryptophan, which was prepared at 50 mM due to its lower solubility. To generate positive amino acid ions, the pH of each solution was lowered below the relevant isoelectric point by the addition of acetic acid. A nanocapillary was pre-filled with an amino acid solution before it was inserted into the vacuum chamber. An extraction voltage Vₑ in the range +260 V to +360 V, applied between the nanocapillary and the extraction electrode, initiated the emission of ionic current of several picoamperes (FIG. 4B). The onset typically occurred abruptly and was accompanied by the measurement of ions striking the instrument's detector at a rate sufficient for clear mass spectra to be collected within minutes to hours.

The mass spectrum of an arginine solution obtained using a nanocapillary ion source with inner tip diameter of 41 nm is shown in FIG. 4D. Five peaks are clearly visible. The peak at 174 m/z corresponds to the singly charged arginine ion (Arg⁺). The higher m/z peaks are all separated by 18 m/z, the shift induced by an additional water molecule. Thus, the other peaks correspond to solvated states of arginine (Arg⁺(H₂O)ₙ), where the solvation number n ranges from 1 to 4.

FIG. 4A is a schematic of conventional electrospray ionization showing the background gas that stimulates evaporation of solvent from droplets and the transfer capillary where significant ion loss occurs. FIG. 4B is a schematic of a nanocapillary ion source showing the liquid-filled nanocapillary tip, the extractor electrode, and Ve applied between them. Inset shows an SEM image of the tip of a pulled quartz nanocapillary with an inner diameter of 30 nm. FIG. 4C is a schematic of the mass spectrometer used in this study. Ion optics comprising an extractor electrode and an Einzel lens extract ions from the liquid meniscus at the ion source and focus them through a quadrupole mass filter and an electrostatic ion bender. The transmitted ions strike a channel electron multiplier detector. FIG. 4D shows a mass spectrum of 100 mM arginine in aqueous solution obtained with a 41 nm inner diameter nanocapillary ion source in our quadrupole mass spectrometer.

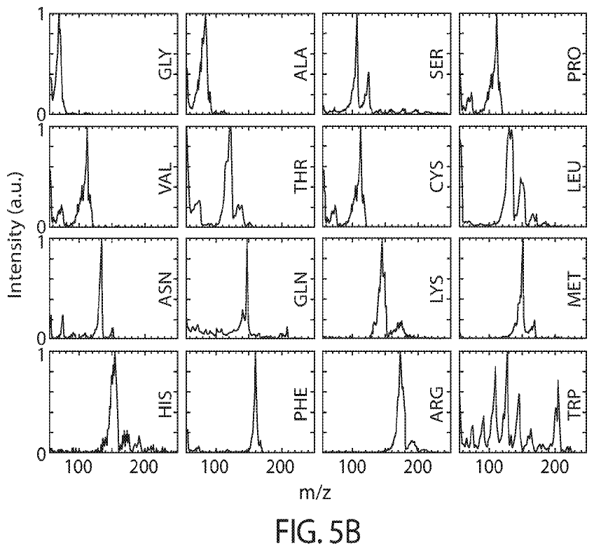

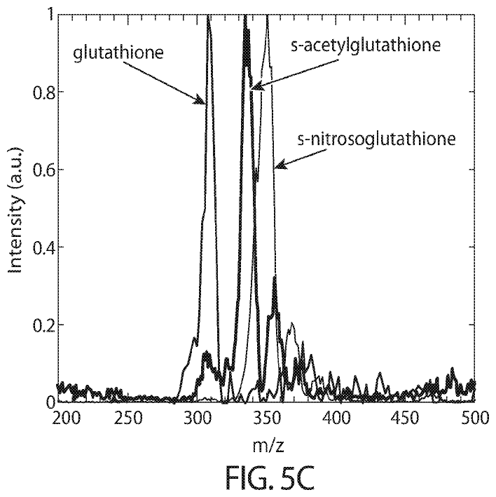

The influence of tip diameter on the arginine mass spectrum is illustrated in FIG. 5A. The mass spectra shown were obtained using nanocapillaries with inner tip diameters of 300 nm, 125 nm, and 20 nm. The largest nanotip produced a broad spectrum of peaks that included the bare arginine ion, eight incrementally hydrated arginine ion clusters, and a peak at 349 m/z corresponding to the arginine dimer ion (Arg·Arg⁺H)⁺. The intermediate-sized tip produced a narrower spectrum that included the bare arginine ion, six incrementally hydrated arginine ion clusters, and a relatively diminished arginine dimer ion peak. The smallest tip primarily produced the bare arginine ion, but attenuated peaks corresponding to the singly and doubly hydrated arginine ion clusters were also visible in the spectrum. Smaller tips tended to produce relatively stronger signals and less noisy spectra than larger tips, as is shown in the baselines of the three spectra in FIG. 5A. Some variance in the distribution of solvation states was observed between nanocapillaries with similar tip sizes (e.g., 20 nm as shown in FIG. 4D compared to 41 nm as shown in FIG. 5A). However, only nanocapillaries with inner tip diameters less than about 65 nm produced spectra where most of the amino acid ions were measured in an unsolvated state.

The nanocapillary source can generate ions of many different amino acids and small peptides for analysis. Solutions inside the nanocapillary were routinely swapped without interrupting the measurement using a fluid delivery system. FIG. 2B shows mass spectra of 16 different aqueous amino acid solutions. Three different nanopocapillaries with inner tip diameters of 20, 25, and 60 nm were used for these measurements. The most prominent peak in every spectrum corresponded to the singly charged and unsolvated amino acid ion. The spectra for glycine, alanine, proline, valine, cysteine, glutamine, and phenylalanine showed no additional peaks which could correspond to solvated amino acid ions. The spectra for serine, threonine, asparagine, lysine, methionine, histidine, arginine, and tryptophan showed a secondary peak 18 m/z to the right of the unsolvated peak, corresponding to the singly hydrated amino acid ion. Leucine showed a third and possibly fourth peak corresponding to higher solvation states. The tryptophan spectrum showed peaks below 200 m/z that were consistent with hydrated states of the hydronium ion and that also appeared in control measurements of aqueous solutions with no amino acid present. Tryptophan had a lower solubility than the other amino acids studied and produced relatively weaker signals. Four amino acids are absent from this example: aspartic acid and glutamic acid were not included as their low isoelectric point would require operation in the negative ion mode, isoleucine is indistinguishable from leucine based on m/z so it was not studied, and tyrosine's low solubility resulted in poor emission characteristics.

Mass spectra of glutathione and two chemically modified variants, s-nitrosoglutathione, and s-acetylglutathione, are shown in FIG. 5C. Glutathione is a tripeptide with biological significance, and the chemically modified forms correspond to common post-translational modifications. The peptides were each analyzed in aqueous solutions at 100 mM concentration with the pH set between 3.1 and 3.9 by the addition of acetic acid. Nanocapillaries with 20 nm inner diameter tips were used to generate the ions. The glutathione spectrum showed a single peak at 307 m/z, which corresponded to the singly charged, unsolvated glutathione ion. The spectra of s-acetylglutathione and s-nitrosoglutathione each showed a dominant peak at 349 m/z and 336 m/z, respectively, corresponding to the singly charged, unhydrated peptide ions; the spectra also showed two progressively smaller peaks 18 and 36 m/z to the right of the dominant peaks, corresponding to singly and doubly hydrated peptide ions, respectively.

FIG. 5A shows a mass spectrum of 100 mM arginine solution in H₂O using nanocapillary ion sources with 3 different inner tip diameters. FIG. 5B shows a gallery of 16 amino acid mass spectra, ordered from top left to bottom right by mass. All experiments were carried out using nanocapillaries with 20-60 nm inner tip diameters. FIG. 5C shows an overlaid mass spectra of glutathione and two of its PTM variants, s-nitrosoglutathione and s-acetylglutathione.

The conventional electrospray mechanism, illustrated in FIG. 4A, was ruled out as the main source of measured ions for two reasons. First, nanoscale aqueous droplets in high vacuum only shed a small fraction of their mass before the evaporation process freezes due to latent heat loss. The instrument described in this example lacked the background gas needed to sustain the evaporation of solvent and the emission of ions in an electrospray process. Second, the small size of the nanocapillaries used in this example restricted the fluid flow rate leaving the tip to 0.1 nL/min or less: at least three orders of magnitude lower than the minimum flow rate needed to sustain a stable cone-jet.

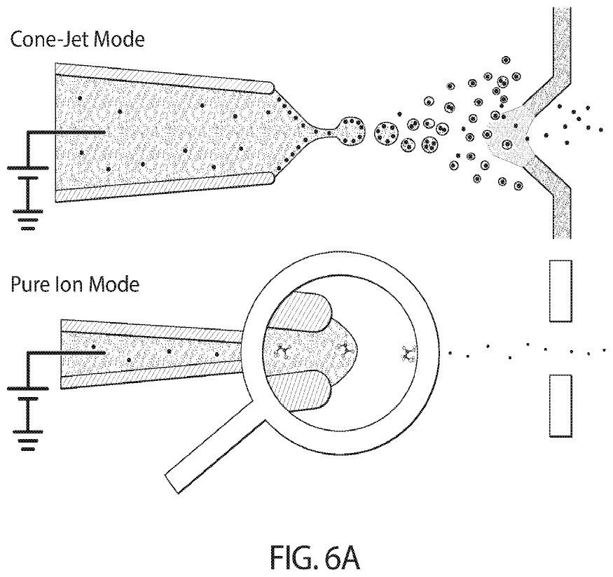

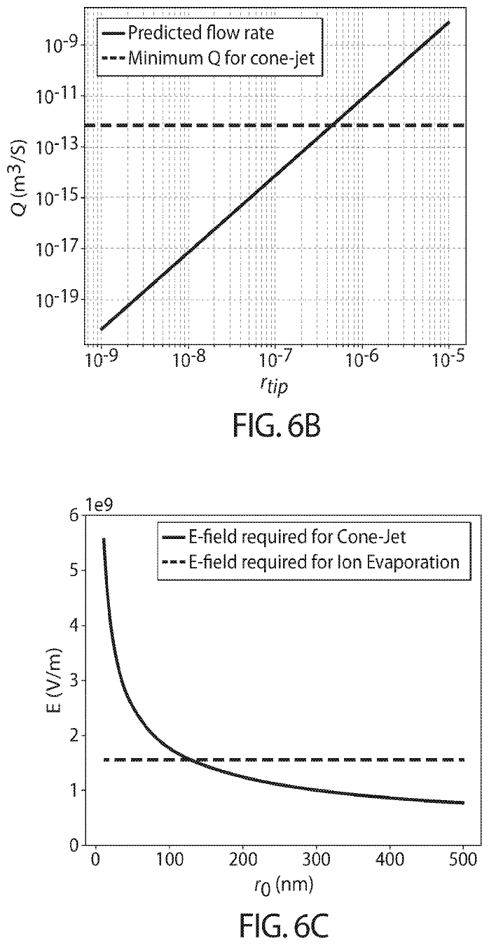

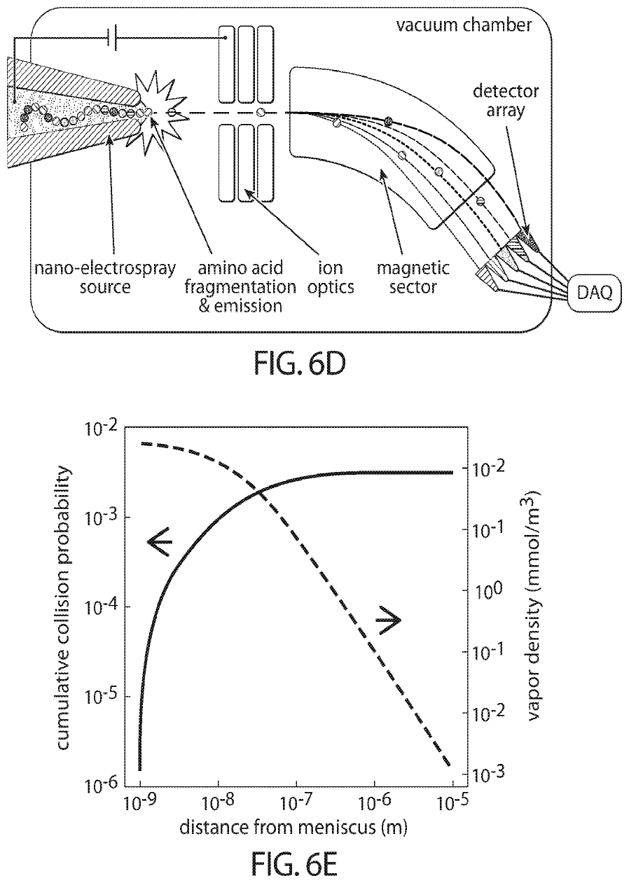

Instead, it is believed that ions were emitted directly from the meniscus at the tip of the ion source by ion evaporation. FIG. 4B is an illustration of pure ion mode emission. The theory of ion emission from Taylor cones predicts that the pure ion mode dominates when the ratio of the liquid conductivity to the flow rate, K/Q, is large. In liquid metals and ionic liquids, this is achieved with a very high conductivity, K. As presented in this example, the same effect is achieved with a very small flow rate, Q. The size of the nanocapillaries chokes off the flow of liquid, restricting the flow rate to less than 0.1 nL/min. FIG. 6B shows the predicted flow rate through the nanocapillaries presented in this example as a function of the tip radius. The inability to form a stable cone-jet due to insufficient flow results in a closed meniscus with high curvature. At such a meniscus, the local electric fields can reach values greater than or equal to 1 V/nm, which is sufficiently high for ion evaporation to occur. FIG. 6C shows the characteristic maximum electric field at the surface of a cone jet as a function of r₀. This field scales as r^{-1/2}, while the field necessary for ion evaporation is independent of r₀. As a result, ion evaporation is expected to be the dominant ion emission mechanism when r₀ is very small.

In conjunction with a mechanism to progressively cleave amino acids from a peptide, a single-molecule protein sequencing mass spectrometer was envisioned, as shown in FIG. 6D. Proteins unfold near the tip of a nanocapillary, likely due to interactions between positively charged protein sites and negatively charged silanol groups on the capillary surface. The unfolded protein can be fragmented into individual amino acids or small peptides by photolysis inside of a nanocapillary ion source, which can then be emitted by ion evaporation. The ions are focused by a set of ion optics and pass through a magnetic sector which separates them by their mass-to-charge ratio. The ions strike an array of electron multiplier detectors, and the original sequence can be reconstructed by considering the timing and location of each ion detection event. 19 detectors can be used to distinguish all of the amino acids excluding leucine/isoleucine. Electron multipliers conventionally used in mass spectrometry can detect ions at a rate of about 100 MHZ.

FIG. 6A is a diagram comparing cone-jet mode ion emission of conventional electrospray with pure ion mode emission from a nanocapillary. FIG. 6B shows a theoretical prediction of flow rate through a tapered capillary as a function of the tip inner radius using a truncated cone model. FIG. 6C shows a theoretical prediction of the characteristic electric field in the transition region of a cone-jet at the tip of a capillary as a function of the capillary tip inner radius, compared with the predicted electric field necessary to achieve ion evaporation from water. FIG. 6D is a schematic of an envisioned mass spectrometer capable of single molecule protein sequencing. FIG. 6E shows a theoretical calculation of the cumulative probability that an emitted amino acid will collide with an evaporated water molecule or background gas molecule as a function of distance from the meniscus. The dashed line shows the calculated maximum possible water vapor density as a function of the distance from the meniscus, calculated using the Hertz-Knudson model with an evaporation coefficient of 1.

The device in this example measured stable emission currents as low as 10 pA, corresponding to an ion emission rate of 60 MHz. As a result, the detectors are fast enough to identify every single ion emitted by the nanocapillary ion source. This sequencing strategy depends on the preservation of the sequence through the fragmentation, emission, and mass separation stages.

As shown in this example, nanocapillary ion sources are able to emit singly charged, unsolvated, biomolecular ions directly into high vacuum. 16 of 20 proteinogenic amino acids, as well as glutathione, s-nitrosoglutathione, and s-acetylglutathione were measured by mass spectrometry to be emitted predominantly in an unsolvated state.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an.” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A mass spectrometer, comprising:
an ion source comprising a capillary and an electrode proximate the capillary, wherein the capillary comprises an opening having a cross-sectional dimension of less than 125 nm;
a magnetic mass filter downstream of the ion source; and
an array of detectors downstream of the magnetic mass filter.

2. The mass spectrometer of claim 1, further comprising a vacuum chamber housing the ion source.

3. The mass spectrometer of claim 2, wherein the vacuum chamber has a pressure of no more than 100 mPa.

4. The mass spectrometer of any one of claim 2 or 3, wherein the vacuum chamber has a pressure of no more than 10 mPa.

5. The mass spectrometer of any one of claims 1-4, wherein the magnetic mass filter comprises a permanent magnet.

6. The mass spectrometer of any one of claims 1-5, further comprising an ion optics downstream of the ion source and upstream of the magnetic mass filter.

7. The mass spectrometer of any one of claims 1-6, furthering comprising an ion bender configured to deflect ions exiting the mass filter to the detector.

8. The mass spectrometer of claim 7, wherein the ion optics comprises at least one Einzel lens.

9. The mass spectrometer of any one of claims 1-8, wherein the mass spectrometer has a temporal resolution of less than or equal to 1 microsecond.

10. The mass spectrometer of any one of claims 1-9, wherein the array of detectors comprises an electron multiplier.

11. The mass spectrometer of any one of claims 1-10, wherein the array of detectors comprises a dynode.

12. The mass spectrometer of any one of claims 1-11, wherein the array of detectors comprises a micro-channel plate array.

13. The mass spectrometer of any one of claims 1-12, wherein the array of detectors comprises a CCD.

14. The mass spectrometer of any one of claims 1-13, wherein the array of detectors comprises a CMOS sensor.

15. The mass spectrometer of any one of claims 1-14, wherein the array of detectors comprises a SQUID.

16. The mass spectrometer of any one of claims 1-15, wherein the opening of the capillary has a cross-sectional dimension of less than 65 nm.

17. The mass spectrometer of any one of claims 1-16, wherein the opening of the capillary has a cross-sectional dimension of less than 50 nm.

18. The mass spectrometer of any one of claims 1-17, wherein the opening of the capillary has a cross-sectional dimension of less than 30 nm.

19. The mass spectrometer of any one of claims 1-18, wherein the opening of the capillary has a cross-sectional dimension of less than 2 nm.

20. The mass spectrometer of any one of claims 1-19, wherein the capillary is tapered at the opening.

21. The mass spectrometer of claim 20, wherein the tapering is at an angle of less than 10°.

22. The mass spectrometer of any one of claim 20 or 21, wherein the tapering is at an angle of less than 5°.

23. The mass spectrometer of any one of claims 1-22, wherein the capillary comprises quartz.

24. The mass spectrometer of any one of claims 1-23, wherein the capillary comprises glass.

25. The mass spectrometer of any one of claims 1-24, wherein the capillary comprises borosilicate glass.

26. The mass spectrometer of any one of claims 1-25, wherein the capillary comprises a plastic.

27. The mass spectrometer of any one of claims 1-26, wherein the capillary comprises a metal.

28. The mass spectrometer of any one of claims 1-27, wherein the capillary comprises a semiconductor.

29. The mass spectrometer of any one of claims 1-28, wherein the capillary comprises a carbon nanotube.

30. The mass spectrometer of any one of claims 1-29, wherein the capillary comprises a boron nitride nanotube.

31. The mass spectrometer of any one of claims 1-30, wherein the capillary has an aspect ratio of length to cross-sectional dimension of greater than or equal to 100.

32. The mass spectrometer of any one of claims 1-31, wherein the capillary has an aspect ratio of length to cross-sectional dimension of greater than or equal to 1,000.

33. The mass spectrometer of any one of claims 1-32, wherein the capillary has an aspect ratio of length to cross-sectional dimension of greater than or equal to 10,000.

34. The mass spectrometer of any one of claims 1-33, wherein the capillary has a cross-sectional dimension of less than 100 nm.

35. The mass spectrometer of any one of claims 1-34, wherein the capillary has a cross-sectional dimension of less than 60 nm.

36. The mass spectrometer of any one of claims 1-35, wherein the electrode defines a center opening.

37. The mass spectrometer of claim 36, wherein the center opening of the electrode has a cross-sectional dimension of less than 5 cm.

38. The mass spectrometer of any one of claim 36 or 37, wherein the center opening of the electrode has a cross-sectional dimension of less than 1 cm.

39. The mass spectrometer of any one of claims 36-38, wherein the center opening of the electrode is larger than the opening of the capillary.

40. The mass spectrometer of any one of claims 36-39, wherein the center opening of the electrode is at least 5 times larger than the opening of the capillary.

41. The mass spectrometer of any one of claims 36-40, wherein the center opening of the electrode is at least 10 times is larger than the opening of the capillary.

42. The mass spectrometer of any one of claims 1-41, wherein the electrode comprises steel.

43. The mass spectrometer of any one of claims 1-42, wherein the electrode is annular.

44. The mass spectrometer of any one of claims 1-43, wherein the electrode has a cross-sectional dimension of less than 5 cm.

45. The mass spectrometer of any one of claims 1-44, wherein the electrode is positioned within 10 mm of the opening of the capillary.

46. The mass spectrometer of any one of claims 1-45, wherein the electrode is positioned within 5 mm of the opening of the capillary.

47. The mass spectrometer of any one of claims 1-46, wherein the electrode is positioned within 2 mm of the opening of the capillary.

48. The mass spectrometer of any one of claims 1-47, wherein the electrode is positioned around the capillary.

49. The mass spectrometer of any one of claims 1-48, wherein the electrode is positioned in front of the opening of the capillary.

50. The mass spectrometer of any one of claims 1-49, wherein an imaginary line passing through centers of cross-sections of the capillary passes through the center opening of the electrode.

51. The mass spectrometer of any one of claims 1-50, wherein the electrode and the capillary has an interior connected to a voltage source.

52. The mass spectrometer of claim 51, wherein the voltage source is capable of producing a voltage between the electrode and the capillary of less 400 V.

53. The mass spectrometer of any one of claim 51 or 52, wherein the voltage source is capable of producing a voltage between the electrode and the capillary of less 360 V.

54. The mass spectrometer of any one of claims 51-53, wherein the voltage source is capable of producing a voltage between the electrode and the capillary of at least 80 V.

55. The mass spectrometer of any one of claims 51-54, wherein the voltage source is capable of producing an electric field between the electrode and the capillary having a maximum of less than or equal to 4 V/nm.

56. The mass spectrometer of any one of claims 51-55, wherein the voltage source is capable of producing an electric field between the electrode and the capillary having a maximum of less than or equal to 3 V/nm.

57. The mass spectrometer of any one of claims 51-56, wherein the voltage source is capable of producing an electric field between the electrode and the capillary having a maximum of at least 1.5 V/nm.

58. The mass spectrometer of any one of claims 1-57, wherein the magnetic mass filter has a magnetic filter strength of at least about 0.5 T.

59. The mass spectrometer of any one of claims 1-58, wherein the magnetic mass filter comprises a magnet comprising neodymium.

60. The mass spectrometer of any one of claims 1-59, wherein the magnetic mass filter comprise a yoke comprising iron.

61. The mass spectrometer of any one of claims 1-60, wherein the magnetic mass filter comprises an opening having a first dimension of at least about 5 cm.

62. The mass spectrometer of any one of claims 1-61, wherein the magnetic mass filter comprises an opening having a second dimension of at least about 1 cm.

63. The mass spectrometer of any one of claims 1-62, wherein the detector is a single-ion detector.

64. A method of sequencing a biopolymer, comprising:
ionizing a biopolymer contained within a fluid into ions or ion clusters;
passing the ions or ion clusters through a magnetic mass filter;
directing the ions or ion clusters to an array of detectors; and
determining a sequence of the biopolymer by determining the ions or ion clusters with the array of detectors.

65. The method of claim 64, wherein the biopolymer is a protein.

66. The method of claim 65, comprising ionizing amino acids of the protein at a rate of at least 1 amino acid per microsecond.

67. The method of any one of claims 64-66, wherein the biopolymer is a nucleic acid.

68. The method of any one of claim 67, comprising ionizing bases of the nucleic acid at a rate of at least 1 base per microsecond.

69. The method of any one of claim 67 or 68, comprising ionizing bases of the nucleic acid at a rate of at least 10 base per microsecond.

70. The method of any one of claims 67-69, comprising ionizing bases of the nucleic acid at a rate of at least 100 base per microsecond.

71. The method of any one of claims 64-70, wherein the ions or ion clusters have at an overall ion transmission efficiency of greater than or equal to about 0.8.

72. The method of any one of claims 64-71, wherein the ions or ion clusters are produced at a rate of greater than or equal to 1 ion or ion cluster/microsecond to 100 ions or ion clusters/microsecond.

73. The method of any one of claims 64-72, wherein the time interval between a molecule exiting as ions or ion clusters proximate the opening and detection of the ions or ion clusters at the array of detectors is greater than or equal to 10 microseconds and less than or equal to 100 microseconds.

74. The method of any one of claims 64-73, wherein the array of detectors comprises an electron multiplier.

75. The method of any one of claims 64-74, wherein the array of detectors comprises a dynode.

76. The method of any one of claims 64-75, wherein the array of detectors comprises a micro-channel plate array.

77. The method of any one of claims 64-76, wherein the array of detectors comprises a CCD.

78. The method of any one of claims 64-77, wherein the array of detectors comprises a CMOS sensor.

79. The method of any one of claims 64-78, wherein the ions or ion clusters may be detected at a time resolution of better than 100 nanoseconds.

80. The method of any one of claims 64-79, wherein ionizing a biopolymer contained within a fluid into ions or ion clusters comprises ionizing the biopolymer into single ions.

81. The method of any one of claims 64-80, wherein passing the ions or ion clusters through a magnetic mass filter comprises passing single ions through a magnetic mass filter.

82. The method of any one of claims 64-81, wherein directing the ions or ion clusters to an array of detectors comprises directing the single ions to an array of detectors.

83. The method of any one of claims 64-82, wherein determining the ions or ion clusters with the array of detector comprises determining the single ions with the array of detector.

84. A method of sequencing a biopolymer, comprising:
passing a fluid comprising a biopolymer into a capillary defining an opening;
applying an electric field to ionize the biopolymer proximate the opening to produce ions or ion clusters;
passing the ions or ion clusters directly into an environment having a pressure of no more than 100 mPa;
passing the ions or ion clusters through a magnetic mass filter;
directing the ions or ion clusters to an array of detectors; and
determining a sequence of the biopolymer by determining the ions or ion clusters with the array of detectors.

85. The method of claim 84, wherein the ions or ion clusters have at an overall ion transmission efficiency of greater than or equal to about 0.8.

86. The method of any one of claim 84 or 85, wherein the ions or ion clusters are produced at a rate of greater than or equal to 1 ion or ion cluster per microsecond to 100 ions or ion clusters per microsecond.

87. The method of any one of claims 84-86, wherein the time interval between a molecule exiting as ions or ion clusters proximate the opening and detection of the ions or ion clusters at the array of detectors is greater than or equal to 10 microseconds and less than or equal to 100 microseconds.

88. The method of any one of claims 84-87, wherein the array of detectors comprises an electron multiplier.

89. The method of any one of claims 84-88, wherein the array of detectors comprises a dynode.

90. The method of any one of claims 84-89, wherein the array of detectors comprises a micro-channel plate array.

91. The method of any one of claims 84-90, wherein the array of detectors comprises a CCD.

92. The method of any one of claims 84-91, wherein the array of detectors comprises a CMOS sensor.

93. A method of determining a concentration, comprising:
ionizing molecules from a fluid as ions or ion clusters;
passing the ions or ion clusters through a magnetic mass filter;
directing the ions or ion clusters to an array of detectors; and
determining a concentration of the molecules in the fluid by determining the ions or ion clusters with the array of detectors.

94. A method, comprising:
ionizing molecules from a fluid as ions or ion clusters;
passing at least 50% of the ions or ion clusters through a magnetic mass filter; and
directing the ions or ion clusters to a detector.

95. The method of claim 94, wherein the detector is one detector of an array of detectors.

96. The method of any one of claim 94 or 95, comprising passing at least 70% of the ions or ion clusters through a magnetic mass filter.

97. The method of any one of claims 94-96, comprising passing at least 80% of the ions or ion clusters through a magnetic mass filter.

98. A method, comprising:
ionizing molecules from a fluid as ions or ion clusters using an ion source;
passing the ions or ion clusters through a mass filter;
directing the ions or ion clusters to a detector; and
determining a duration between the time the ions or ion clusters leave the ion source and the time the ions or ion clusters arrives at the detector.

99. The method of claim 98, comprising determining the duration at a time resolution of

100. A mass spectrometer, comprising:
an ion source constructed and arranged to produce single ions or ion clusters;
a magnetic mass filter positioned to receive the single ions or ion clusters from the ion source;
a pump able to create a pressure less than 100 mPa in an environment positioned between the ion source and the magnetic mass filter; and
an array of detectors positioned to receive the single ions or ion clusters from the magnetic mass filter.

101. A mass spectrometer, comprising:
an ion source;
a magnetic mass filter downstream of the ion source; and
an array of detectors downstream of the magnetic mass filter.

102. The mass spectrometer of claim 101, wherein the ion source comprises a pulsed laser.

103. The mass spectrometer of any one of claim 101 or 102, wherein the ion source comprises a capillary and an electrode proximate the capillary, wherein the capillary comprises an opening having a cross-section of less than 125 nm.

104. A method, comprising:
ionizing molecules using an ion source to produce a sequence of ions or ion clusters;
passing the sequence of ions or ion clusters through a mass filter; and
directing the sequence of ions or ion clusters to an array of detectors, wherein at least 90% of the ions or ion clusters arriving at the array of detectors arrive in sequence.
